(19) United States
(12) Patent Application Publication
(10) Pub. No.: US 2025/0177044 A1
(43) Pub. Date: Jun. 5, 2025

(54) Systems, Methods and Apparatus of Mixed Reality Tumor Resection Planning

(71) Applicant: apoQlar GmbH, Hamburg (DE)

(72) Inventors: Liliana Duarte, Hamburg (DE); Radoslaw Furmaniak, Hamburg (DE); Daniel Jader, Hamburg (DE); Roman Kupalov, Hamburg (DE); Konrad Marciniak, Hamburg (DE); Erke Tellal, Hamburg (DE)

(73) Assignee: apoQlar GmbH, Hamburg (DE)

(21) Appl. No.: 18/530,188

(22) Filed: Dec. 5, 2023

(51) Int. Cl. A61B 34/10; A61B 90/00; G06V 10/26; G06V 10/96; G06V 20/70; G16H 20/40; G16H 30/20; G16H 40/20; G16H 50/30

(52) U.S. Cl. CPC A61B 34/10; A61B 90/37; G06V 10/26; G06V 10/96; G06V 20/70; G16H 20/40; G16H 30/20; G16H 40/20; G16H 50/30; A61B 2090/365; G06V 2201/03

(57) ABSTRACT

Systems, methods and apparatus are provided through which a tumor resection planning tool in mixed reality comprises apparatuses for image registration, synchronous navigation through fused datasets, segmentation and labeling of surgery-related structures, manual correction of segmentation results, and a browser-based tool for clinical validation. In some implementations the tumor resection planning tool generates a step-by-step surgical tumor resection plan adaptable to organs such as liver, pancreas, lung, and brain. In some implementation the tumor resection planning tool displays segmented structures as 3D polygonal objects with customizable textures, transparency, and color. Risk margins offset from tumor surfaces are visualized with user-defined values. In some implementations vessel clip plan tool(s) simulate post-resection blood flow. Users can create, modify, and share resection plans, dividing organ structures based on user-defined plans. In some implementations vessel clip plan tool(s) enables a mixed reality shared experience for remote discussion of surgery plans among tumor board members.

### FIELD

This disclosure relates generally to a mixed reality tumor resection planning tool, and more particularly conducting tumor resection planning in mixed reality.

### BACKGROUND

The surgical removal of tumors from various anatomical structures (e.g. lung, brain or liver) of the body requires the use of tumor resection surgeries. Preoperative planning that is precise and considers the tumor's position, size, and relationship to other anatomical structures is crucial to the success of these operations. However, the constraints and difficulties that currently exist in tumor resection planning techniques frequently have an impact on surgical outcomes.

As an example, hepatectomy, commonly known as liver resection, provides a particularly difficult scenario for the removal of tumors. Surgery requires sophisticated planning since the liver is a complicated organ with numerous lobes, arteries, and bile ducts. The surgical approach and resection margins might be significantly impacted by the placement of the tumor in respect to important structures, such as major blood arteries or bile ducts.

Conventional approaches for tumor resection planning typically involve the interpretation of 2D medical imaging data, such as CT scans or MRI scans on flat screen displays. Surgeons try to mentally reconstruct the complex 3D anatomy of the target organ and the plan the surgery accordingly. However, because of the subjectivity of this process and the potential for variation in how spatial relationships are interpreted, there is a greater chance that the tumor won't completely resected or that crucial vascular structures would be harmed.

Additionally, lack of interaction and teamwork in conventional methods of planning hinder thorough analysis of various surgical procedures and the transfer of knowledge. When trying to communicate and modify their surgical plans, surgeons frequently run into difficulties, especially when working remotely with colleagues.

### SUMMARY

The above-mentioned shortcomings, disadvantages and problems are addressed herein, which will be understood by reading and studying the following specification.

The present disclosure provides better tumor resection planning tool created especially for difficult instances like liver resection to solve the above-mentioned issues. Utilizing cutting-edge mixed reality technology can greatly increase visualization, interactivity, and collaboration, improving the results of surgeries.

This disclosure is a comprehensive tumor resection planning tool in mixed reality that revolutionizes the surgical planning process for various organs, including the liver, pancreas, lung, and brain. The tool offers a step-by-step workflow that guides surgeons through the entire planning process, providing intuitive and interactive features.

This disclosure enables the registration or fusion of CT images in different phases and/or the registration of CT and MR images, allowing for a comprehensive understanding of the tumor's location and its relationship with surrounding structures. Surgeons can seamlessly navigate through synchronized slices in the fused datasets, enhancing their spatial perception and accuracy during planning.

To facilitate accurate surgical planning, the tool incorporates sophisticated segmentation techniques to digitally label surgery-related structures, such as parenchyma, hepatic vein, portal vein, and tumors. Surgeons can review and manually correct the segmentation results using a browser-based segmentation tool, ensuring the final clinical validation of the segmentation.

This disclosure presents segmented structures as 3D polygonal objects with predefined textures, enhancing the surgeon's perception of the relationship between anatomical structures. Users can adjust the visualization of objects in terms of transparency and color to meet their specific needs.

Furthermore, this disclosure provides an intuitive interface for surgeons to define risk margins offset from the tumor surface, with the flexibility to adjust margin values based on user preferences. Vessel clipping planning tools simulate blood flow and the behavior of vascular structures post-resection, aiding in preoperative evaluation and risk assessment.

Surgeons can create, move, remove, and modify tumor resection planes around target tumors, precisely dividing the target organ structure into remaining and to be resected parts. The tool calculates and displays the remaining target organ volume based on the division, assisting surgeons in assessing the impact of the resection.

This disclosure allows for the saving and loading of surgery plans, including divided polygonal objects, remaining organ volume, and vessel clipping information. Additionally, the tool facilitates a shared mixed reality experience, enabling remote discussion of the surgery plan with other tumor board members.

In summary, this tumor resection planning tool in mixed reality provides surgeons with a comprehensive and intuitive solution for precise surgical planning across multiple organs. It leverages advanced visualization, interactive segmentation, risk assessment, and collaborative features to enhance surgical outcomes, improve patient care, and advance the field of tumor resection.

In one aspect, a tumor resection planning tool in mixed reality includes, an apparatus that is operable to register or fuse a set of computed tomography images in different phases and/or register computed tomography and magnetic resonance images, an apparatus that is operable to synchronously navigate through slices in fused datasets, an apparatus that is operable to segment and digitally label surgery-related structures, for example in Hepatectomy (Liver Resection) include parenchyma, hepatic vein, portal vein, and tumors, from fused DICOMs, yielding segmentation results, an apparatus that is operable to review and manually correct the segmentation results through a browser-based segmentation tool for a final clinical validation of the segmentation results, an apparatus that is operable to provide a step-by-step surgical tumor resection plan workflow that is adaptable to different organs such as a liver, pancreas, a lung, and a brain, an apparatus that is operable to display a set of segmented structures as 3D polygonal objects with assigned predefined textures to enhance a perception by a surgeon of relationships between anatomical structures, an apparatus that is operable to adjust a visualization of objects in terms of transparency and color to meet user's custom needs, the apparatus that is operable to adjust the visualization of the objects being operable to the apparatus that is operable to display the set of segmented structures, an apparatus that is operable to display different levels of risk margins offset from a tumor surface, with user-defined margin values that can be changed on demand, an apparatus that is operable to provide vessel clip plan tools to simulate a blood flow and behavior of vascular structures after a resection according to the step-by-step surgical tumor resection plan workflow, an apparatus that is operable to create, move, remove, and modify the step-by-step surgical tumor resection plan workflow around a set of target tumor(s), an apparatus that is operable to divide a target organ structure into remain and to-be-resected parts based on a user-defined resection plan(s) by applying cloud-based boolean mesh operations yielding a division, an apparatus that is operable to offer surgeons an ability to edit the step-by-step surgical tumor resection plan workflow and reapply the division, an apparatus that is operable to indicate a surgery plan includes divided polygonal objects, remain organ volume, and vessel clip information, an apparatus that is operable to provide surgeons an option to load a previously saved surgery plan, and, an apparatus that is operable to provide surgeons a mixed reality shared experience to remotely discuss the surgery plan with other tumor board members.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF DRAWINGS

FIG. 1 is a diagram illustrating a system-level overview of an implementation.

FIG. 1A is a block diagram of a resection result 1A00, according to an implementation.

FIG. 2 is a block diagram of an apparatus to segment an image of a liver, according to an implementation.

FIG. 2A is a block diagram of a liver segmentation object 2A00, according to an implementation.

FIG. 2B is a block diagram of a resection result 2B00, according to an implementation.

FIG. 3 is a block diagram of software classes to segment an image of a liver, according to an implementation.

FIG. 4 is a flowchart of a method of tumor resection planning in mixed reality, according to an implementation.

FIG. 5 is a sample photo representing the users field of view from a mixed reality scene consisting of a medical object hologram with an image of a liver with resected and non-resected parts, according to an implementation.

FIG. 6 is a sample photo representing the users field of view from a mixed reality scene consisting of a medical object hologram with an image of a liver with a non-modified resection plane, according to an implementation.

FIG. 7 is a sample photo representing the users field of view from a mixed reality scene consisting of a medical object hologram with an image of a liver with a liver with a user defined resection plane, according to an implementation.

FIG. 8 is a diagram of an image of a liver with virtual vessel clips, according to an implementation.

FIG. 9 is a diagram of an image of a liver with user defined risk margins, according to an implementation.

FIG. 10 is a sample photo representing the users field of view from a mixed reality scene consisting of a medical object hologram with a virtual user interaction menu with a result of resection, according to an implementation.

FIG. 11 is a block diagram of a mixed reality tumor resection planning control computer in which different implementations can be practiced.

FIG. 12 is a block diagram of a mixed reality tumor resection planning control computer in which different implementations can be practiced.

FIG. 13 is a block diagram of a data acquisition circuit of the mixed reality tumor resection planning control computer, according to an implementation.

FIG. 14 is a block diagram of a hardware and operating environment in which different implementations can be practiced.

FIG. 15 is a block diagram of a mixed reality tumor resection planning control mobile device, according to an implementation.

### DETAILED DESCRIPTION OF THE INVENTION

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

The detailed description is divided into five sections. In the first section, a system level overview is described. In the second section, apparatus of implementations are described. In the third section, implementations of methods are described. In the fourth section, a hardware and the operating environment in conjunction with which implementations may be practiced are described. Finally, in the fifth section, a conclusion of the detailed description is provided.

### System Level Overview

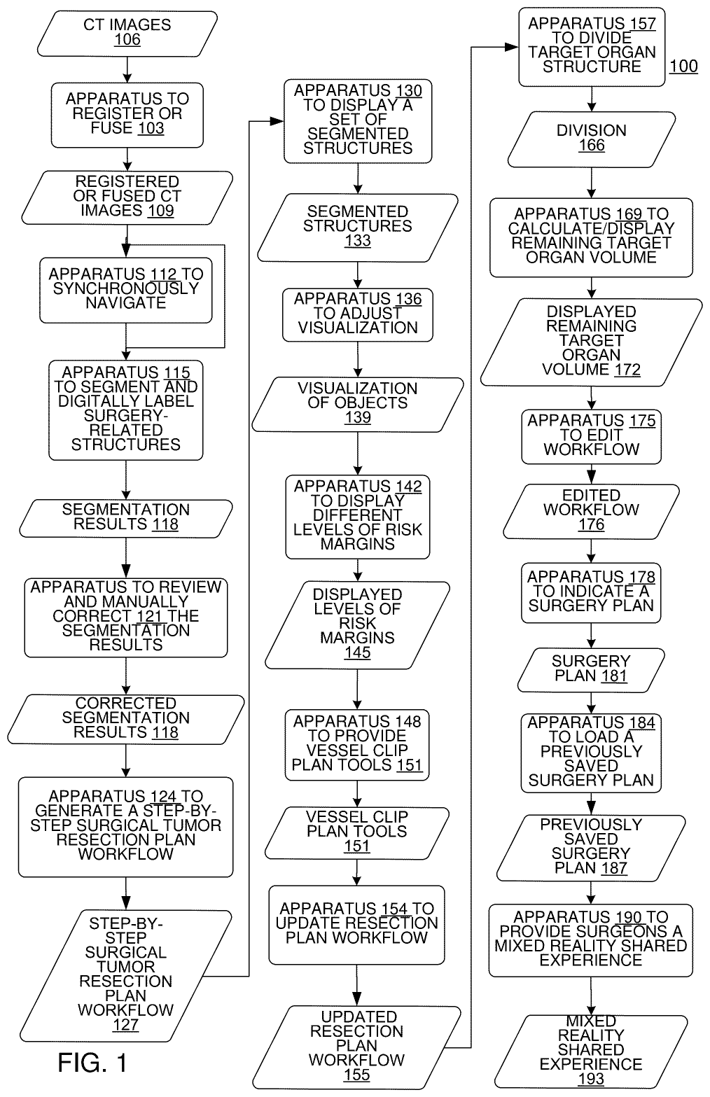

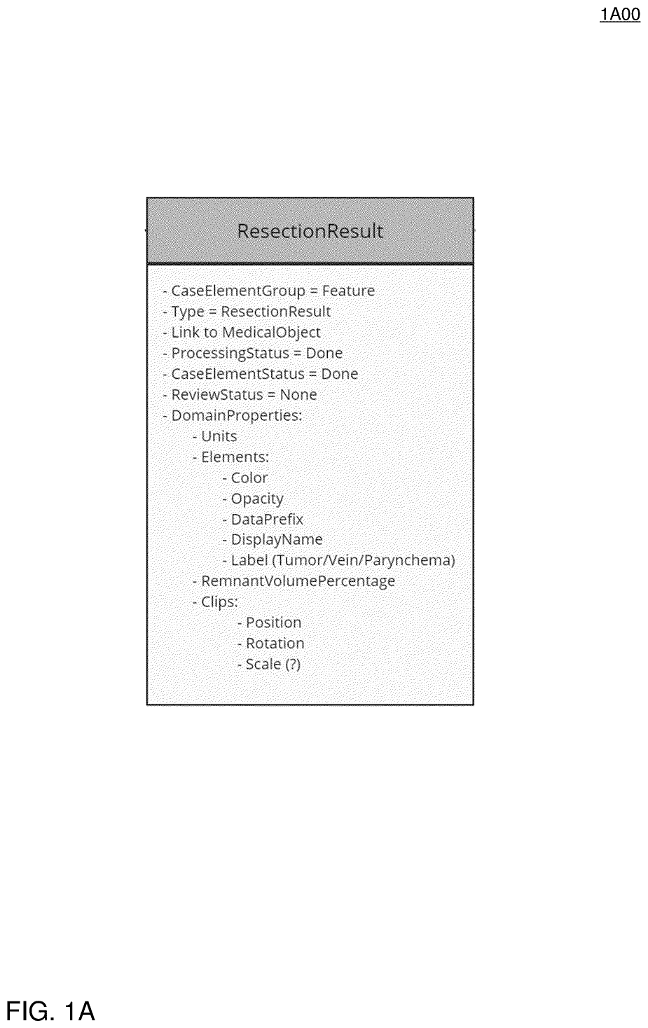

FIG. 1 is a block diagram of a tumor resection planning tool in mixed reality apparatus 100, according to an implementation, according to an implementation. The system level overview of the operation of an implementation is described in this section of the detailed description.

Apparatus 100 includes an apparatus or component 103 that is operable to register or fuse a set of computed tomography images 106 in different phases and/or register computed tomography and magnetic resonance images, according to an implementation, yielding registered or fused computed tomography images 109.

Apparatus 100 also includes an apparatus or component 112 that is operable to synchronously navigate through slices in the fused images 109. The apparatus or component 112 that is operable to synchronously navigate is operably coupled to the apparatus or component 103 that is operable to register or fuse the set of registered of fused computed tomography images 109, according to an implementation.

Apparatus 100 also includes an apparatus or component 115 that is operable to segment and digitally label surgery-related structures. For example, in Hepatectomy (Liver Resection) include parenchyma, hepatic vein, portal vein, and tumors, from fused DICOMs, yielding segmentation results 118. The apparatus or component 115 that is operable to segment is operably coupled to the apparatus or component 112 that is operable to synchronously navigate, according to an implementation.

Apparatus 100 also includes an apparatus or component 121 that is operable to review and manually correct the segmentation results 118 through a browser-based segmentation tool for a final clinical validation of the segmentation results. The apparatus or component 121 that is operable to review and manually correct segmentation results that is operably coupled to the apparatus or component 115 that is operable to segment, according to an implementation.

Apparatus 100 also includes an apparatus or component 124 that is operable to provide a step-by-step surgical tumor resection plan workflow 127 adaptable to different organs such as a liver, pancreas, a lung, and a brain. The apparatus or component 124 that is operable to provide the step-by-step surgical tumor resection plan workflow 127 is operably coupled to the apparatus or component 121 that is operable to review and manually correct segmentation results 118, according to an implementation.

Apparatus 100 also includes an apparatus or component 130 that is operable to display a set of segmented structures 133 as 3D polygonal objects with assigned predefined textures to enhance a perception by a surgeon of relationships between anatomical structures. The apparatus or component 130 that is operable to display the set of the segmented structures 133 is operably coupled to the apparatus or component 124 that is operable to provide the step-by-step surgical tumor resection plan workflow 127, according to an implementation.

Apparatus 100 also includes an apparatus or component 136 that is operable to adjust a visualization of objects 139 in terms of transparency and color to meet user's custom needs. The apparatus or component 136 that is operable to adjust the visualization of the objects 139 is operably coupled to the apparatus or component 130 that is operable to display the set of segmented structures 133, according to an implementation.

Apparatus 100 also includes an apparatus or component 142 that is operable to display different levels of risk margins 145 that are offset from a tumor surface, with user-defined margin values that can be changed on demand. The apparatus or component 142 that is operable to display the different levels of the risk margins 145 is operably coupled to the apparatus or component 136 that is operable to adjust the visualization of objects 139, according to an implementation.

Apparatus 100 also includes an apparatus or component 148 that is operable to provide vessel clip plan tools 151 to simulate a blood flow and behavior of vascular structures after a resection according to the step-by-step surgical tumor resection plan workflow 127. The apparatus or component 148 that is operable to provide vessel clip plan tools 151 is operably coupled to the apparatus or component 142 that is operable to display the different levels of the risk margins 145, according to an implementation.

Apparatus 100 also includes an apparatus or component 154 that is operable to create, move, remove, and modify the step-by-step surgical tumor resection plan workflow 127 around a set of target tumor(s). The apparatus or component 154 that is operable to create, move, remove, and modify step-by-step surgical tumor resection plan workflow 127 is operably coupled to the apparatus or component 148 that is operable to provide vessel clip plan tools 151, according to an implementation.

Apparatus 100 also includes an apparatus or component 157 that is operable to divide a target organ structure 160 into remain and to-be-resected parts based on a user-defined resection plan(s) 163 by applied cloud-based boolean mesh operations, yielding a division 166. The apparatus or component 157 that is operable to divide a target organ structure 160 is operably coupled to the apparatus or component 154 that is operable to create, move, remove, and modify the tumor resection plan workflow 127, according to an implementation.

Apparatus 100 also includes an apparatus or component 169 that is operable to calculate and display remaining target organ volume 172 based on the division 166. The apparatus or component 169 that is operable to calculate and display remaining target organ volume 172 is operably coupled to the apparatus or component 157 that is operable to divide the target organ structure 160.

Apparatus 100 also includes an apparatus or component 175 that is operable to offer surgeons an ability to edit the step-by-step surgical tumor resection plan workflow 127 and reapply the division 166. The apparatus or component 175 that is operable to offer surgeons an ability to edit the step-by-step surgical tumor resection plan workflow 127 is operably coupled to the apparatus or component 169 that is operable to calculate and display remaining target organ volume 172, according to an implementation.

Apparatus 100 also includes an apparatus or component 178 that is operable to indicate a surgery plan 181 includes divided polygonal objects, remain organ volume, and vessel clip information. The apparatus or component 178 that is operable to indicate a surgery plan 181 is operably coupled to the apparatus or component 178 that is operable to offer surgeons the ability to edit the step-by-step surgical tumor resection plan workflow 127, according to an implementation.

Apparatus 100 also includes an apparatus or component 184 that is operable to provide surgeons an option to load a previously saved surgery plan 187. The apparatus or component 184 that is operable to provide surgeons an option to load a previously saved surgery plan 187 is operably coupled to the apparatus or component 178 that is operable to indicate the surgery plan 181.

Apparatus 100 also includes an apparatus or component 190 that is operable to provide surgeons a mixed reality shared experience 193 to remotely discuss the surgery plan with other tumor board members. The apparatus or component 190 that is operable to provide surgeons a mixed reality shared experience 193 is operably coupled to the apparatus or component 184 that is operable to provide surgeons the option to load the previously saved surgery plan 187.

While the apparatus 100 is not limited to any particular apparatus or component 103 that is operable to register or fuse a set of computed tomography images 106 in different phases and/or register computed tomography and magnetic resonance images, registered or fused computed tomography images 109, apparatus or component 112 that is operable to synchronously navigate through slices in the fused images 109, apparatus or component 112 that is operable to synchronously navigate, apparatus or component 103 that is operable to register or fuse the set of registered of fused computed tomography images 109, apparatus or component 115 that is operable to segment and digitally label surgery-related structures, yielding segmentation results 118, apparatus or component 115 that is operable to segment, apparatus or component 112 that is operable to synchronously navigate, apparatus or component 121 that is operable to review and manually correct the segmentation results 118 through a browser-based segmentation tool for a final clinical validation of the segmentation results, apparatus or component 121 that is operable to review and manually correct segmentation results 118, apparatus or component that is operable to segment, apparatus or component 124 that is operable to provide a step-by-step surgical tumor resection plan workflow 127, apparatus or component 124 that is operable to provide the step-by-step surgical tumor resection plan workflow 127, apparatus or component 121 that is operable to review and manually correct segmentation results 118, apparatus or component 130 that is operable to display a set of segmented structures 133 as 3D polygonal objects with assigned predefined textures to enhance a perception by a surgeon of relationships between anatomical structures, apparatus or component 130 that is operable to display the set of the segmented structures 133, apparatus or component 124 that is operable to provide the step-by-step surgical tumor resection plan workflow 127, apparatus or component 136 that is operable to adjust a visualization of objects 139 in terms of transparency and color to meet user's custom needs, apparatus or component 136 that is operable to adjust the visualization of the objects139, apparatus or component 130 that is operable to display the set of segmented structures 133, apparatus or component 142 that is operable to display different levels of risk margins 145 that are offset from a tumor surface, with user-defined margin values that can be changed on demand, apparatus or component 142 that is operable to display the different levels of the risk margins 145, apparatus or component 136 that is operable to adjust the visualization of objects 139, apparatus or component 148 that is operable to provide vessel clip plan tools 151 to simulate a blood flow and behavior of vascular structures after a resection according to the step-by-step surgical tumor resection plan workflow 127, apparatus or component 148 that is operable to provide vessel clip plan tools 151, apparatus or component 142 that is operable to display the different levels of the risk margins 145, apparatus or component 154 that is operable to create, move, remove, d modify the step-by-step surgical tumor resection plan workflow 127 around a set of target tumor(s), apparatus or component 154 that is operable to create, move, remove, d modify step-by-step surgical tumor resection plan workflow 127, apparatus or component 148 that is operable to provide vessel clip plan tools 151, apparatus or component that 157 is operable to divide a target organ structure 160 into remain and to-be-resected parts based on a user-defined resection plan(s) 163 by applied cloud-based boolean mesh operations, yielding a division 166, apparatus or component 157 that is operable to divide a target organ structure 160, apparatus or component 154 that is operable to create, move, remove, d modify the tumor resection plan workflow 127, apparatus or component 169 that is operable to calculate and display remaining target organ volume 172 based on the division 166, apparatus or component 169 that is operable to calculate and display remaining target organ volume 172, apparatus or component 157 that is operable to divide the target organ structure 160, apparatus or component 175 that is operable to offer surgeons an ability to edit the step-by-step surgical tumor resection plan workflow 127 and reapply the division 166, apparatus or component 175 that is operable to offer surgeons an ability to edit the step-by-step surgical tumor resection plan workflow 127, apparatus or component 169 that is operable to calculate and display remaining target organ volume 172, apparatus or component 178 that is operable to indicate a surgery plan 181 includes divided polygonal objects, remain organ volume, d vessel clip information, apparatus or component 178 that is operable to indicate a surgery plan 181, apparatus or component 178 that is operable to offer surgeons the ability to edit the step-by-step surgical tumor resection plan workflow 127, apparatus or component 184 that is operable to provide surgeons an option to load a previously saved surgery plan 187, apparatus or component 184 that is operable to provide surgeons an option to load a previously saved surgery plan 187, apparatus or component 178 that is operable to indicate the surgery plan 181, apparatus or component 190 that is operable to provide surgeons a mixed reality shared experience 193 to remotely discuss the surgery plan with other tumor board members, apparatus or component 190 that is operable to provide surgeons a mixed reality shared experience 193, apparatus or component 184 that is operable to provide surgeons the option to load the previously saved surgery plan 187, for sake of clarity a simplified apparatus or component 103 that is operable to register or fuse a set of computed tomography images 106 in different phases and/or register computed tomography and magnetic resonance images, registered or fused computed tomography images 109, apparatus or component 112 that is operable to synchronously navigate through slices in the fused images 109, apparatus or component 112 that is operable to synchronously navigate, apparatus or component 103 that is operable to register or fuse the set of registered of fused computed tomography images 109, apparatus or component 115 that is operable to segment and digitally label surgery-related structures, yielding segmentation results 118, apparatus or component 115 that is operable to segment, apparatus or component 112 that is operable to synchronously navigate, apparatus or component 121 that is operable to review and manually correct the segmentation results 118 through a browser-based segmentation tool for a final clinical validation of the segmentation results, apparatus or component 121 that is operable to review and manually correct segmentation results 118, apparatus or component that is operable to segment, apparatus or component 124 that is operable to provide a step-by-step surgical tumor resection plan workflow 127, apparatus or component 124 that is operable to provide the step-by-step surgical tumor resection plan workflow 127, apparatus or component 121 that is operable to review and manually correct segmentation results 118, apparatus or component 130 that is operable to display a set of segmented structures 133 as 3D polygonal objects with assigned predefined textures to enhance a perception by a surgeon of relationships between anatomical structures, apparatus or component 130 that is operable to display the set of the segmented structures 133, apparatus or component 124 that is operable to provide the step-by-step surgical tumor resection plan workflow 127, apparatus or component 136 that is operable to adjust a visualization of objects 139 in terms of transparency and color to meet user's custom needs, apparatus or component 136 that is operable to adjust the visualization of the objects139, apparatus or component 130 that is operable to display the set of segmented structures 133, apparatus or component 142 that is operable to display different levels of risk margins 145 that are offset from a tumor surface, with user-defined margin values that can be changed on demand, apparatus or component 142 that is operable to display the different levels of the risk margins 145, apparatus or component 136 that is operable to adjust the visualization of objects 139, apparatus or component 148 that is operable to provide vessel clip plan tools 151 to simulate a blood flow and behavior of vascular structures after a resection according to the step-by-step surgical tumor resection plan workflow 127, apparatus or component 148 that is operable to provide vessel clip plan tools 151, apparatus or component 142 that is operable to display the different levels of the risk margins 145, apparatus or component 154 that is operable to create, move, remove, d modify the step-by-step surgical tumor resection plan workflow 127 around a set of target tumor(s), apparatus or component 154 that is operable to create, move, remove, d modify step-by-step surgical tumor resection plan workflow 127, apparatus or component 148 that is operable to provide vessel clip plan tools 151, apparatus or component that 157 is operable to divide a target organ structure 160 into remain and to-be-resected parts based on a user-defined resection plan(s) 163 by applied cloud-based boolean mesh operations, yielding a division 166, apparatus or component 157 that is operable to divide a target organ structure 160, apparatus or component 154 that is operable to create, move, remove, d modify the tumor resection plan workflow 127, apparatus or component 169 that is operable to calculate and display remaining target organ volume 172 based on the division 166, apparatus or component 169 that is operable to calculate and display remaining target organ volume 172, apparatus or component 157 that is operable to divide the target organ structure 160, apparatus or component 175 that is operable to offer surgeons an ability to edit the step-by-step surgical tumor resection plan workflow 127 and reapply the division 166, apparatus or component 175 that is operable to offer surgeons an ability to edit the step-by-step surgical tumor resection plan workflow 127, apparatus or component 169 that is operable to calculate and display remaining target organ volume 172, apparatus or component 178 that is operable to indicate a surgery plan 181 includes divided polygonal objects, remain organ volume, d vessel clip information, apparatus or component 178 that is operable to indicate a surgery plan 181, apparatus or component 178 that is operable to offer surgeons the ability to edit the step-by-step surgical tumor resection plan workflow 127, apparatus or component 184 that is operable to provide surgeons an option to load a previously saved surgery plan 187, apparatus or component 184 that is operable to provide surgeons an option to load a previously saved surgery plan 187, apparatus or component 178 that is operable to indicate the surgery plan 181, apparatus or component 190 that is operable to provide surgeons a mixed reality shared experience 193 to remotely discuss the surgery plan with other tumor board members, apparatus or component 190 that is operable to provide surgeons a mixed reality shared experience 193, apparatus or component 184 that is operable to provide surgeons the option to load the previously saved surgery plan 187 are described.

### Apparatus Implementations

In the previous section, a system level overview of the operation of an implementation was described. In this section, the particular apparatus of such an implementation are described by reference to a series of diagrams.

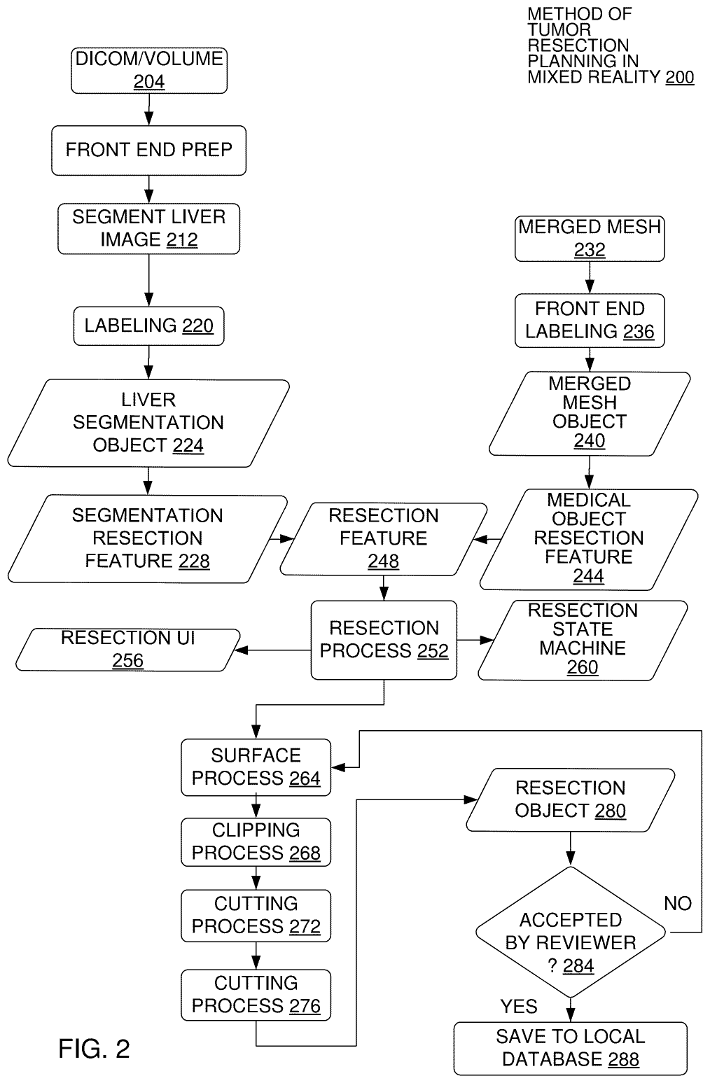

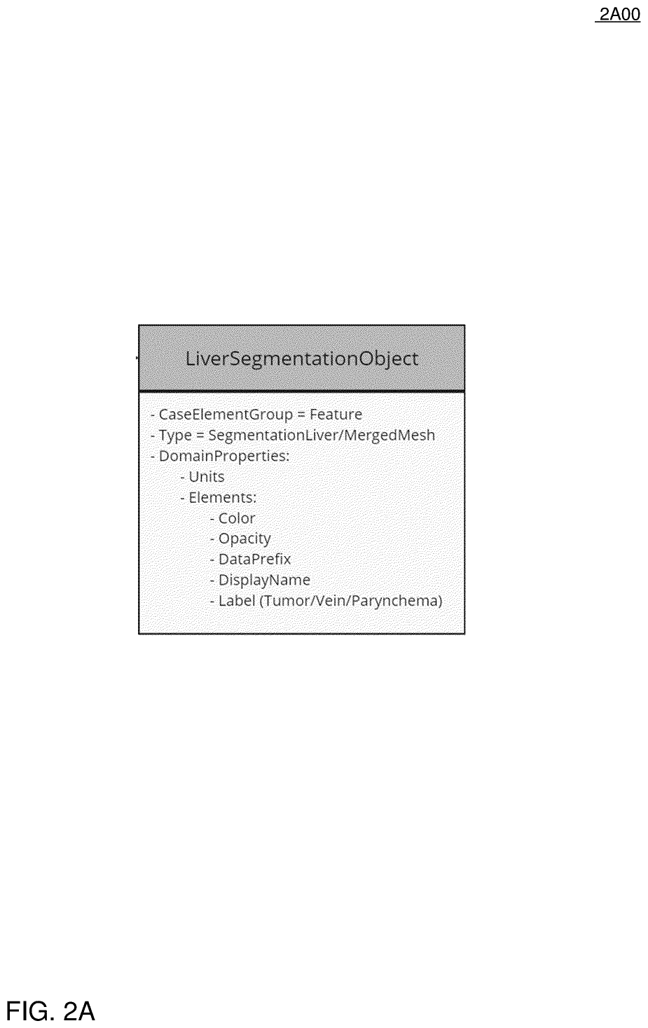

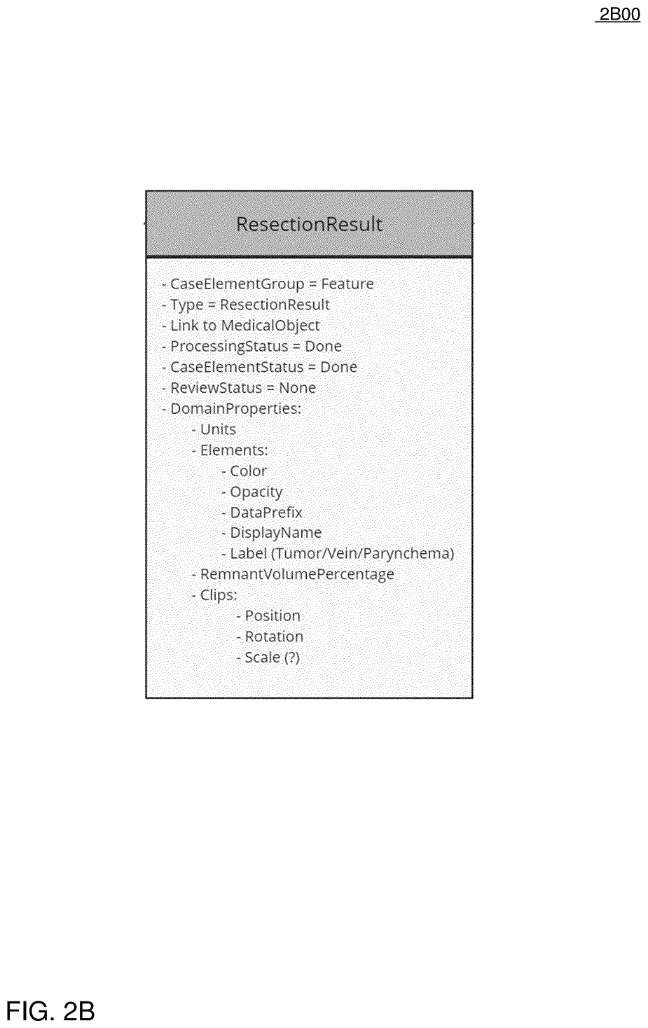

FIG. 2 is a cross section block diagram of apparatus 200 to segment an image of a liver, according to an implementation. Apparatus 200 provides a liver resection object.

Apparatus 200, when a user triggers a liver segmentation feature, a DICOM/volume file 204 is accessed by a front-preparation process 208, in which an operator selects structure(s) to segment, after which a liver image in the DICOM/volume file is segmented into segmentation portion(s) at block 212 in accordance with the user-selected structure(s), and the segmented portion(s) are labeled at block 220, resulting in a liver segmentation object 224 as described in greater detail in FIG. 2A.

Thereafter, a segmentation resection feature 228 is extracted or exported from the liver segmentation object 224.

Apparatus 200 also includes a merged mesh file 232 upon which front-end labeling is performed 236, (such as labeling structures as tumor, vein or Parenchyma) that is initiated when a user opens a model adjustment, yielding a merged mesh object 240, and thereafter a medical object resection feature 244 is extracted or exported from the merged mesh object 240.

Thereafter a resection feature 248 combines the segmentation resection feature 228 and the medical object resection feature 244 and from the resection feature 248, and a resection process 252 yields a resection user interface 256 and a resection state machine 260.

Thereafter, a surface process 264, a clipping process 268, a first cutting process 272 and a second cutting process 276 yields the resection object 280, as described in greater detail in FIG. 2B.

If the resection object 280 is not accepted by a user at block 284, the apparatus 200 continues at the surface process 264, otherwise the resection object 280 is stored or saved to a local or remote database and the apparatus 200 ends.

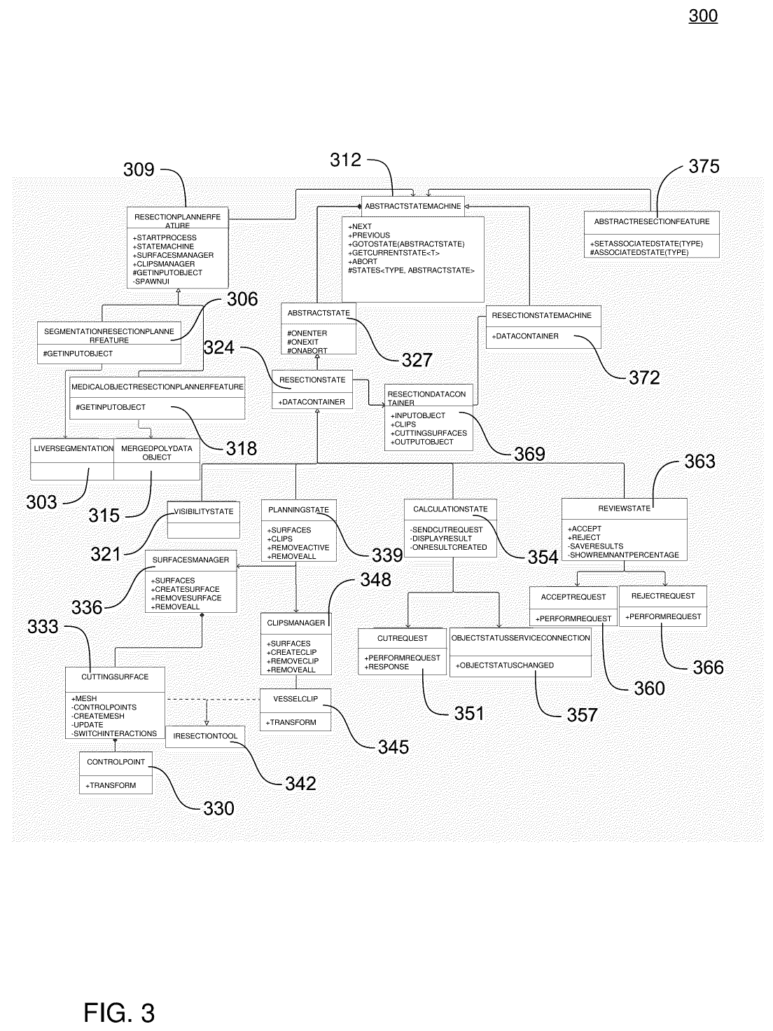

FIG. 3 is a class diagram of liver segmentation classes 300, according to an embodiment.

The FIG. 3 uses the Unified Modeling Language (UML), which is an industry standard language to specify, visualize, construct, and document the object-oriented artifacts of software systems. In FIG. 3, a hollow arrow between classes is used to indicate that a child class below a parent class inherits attributes and methods from the parent class. A class is a prototype of an object that defines data and methods. An object is an instantiation of a class. In addition, a conventional arrow is used to indicate that an object of the class that is depicted above an object of another class is composed of the lower depicted object. Composition defines the attributes of an instance of a class as containing an instance of one or more existing instances of other classes in which the composing object does not inherit from the object(s) of which it is composed.

The liver segmentation classes 300 include a liver segmentation class 303 of which a segmentation resection planner feature class 306 is composed of, and the segmentation resection planner feature class 306 Is a child class of a resection planner feature class 309, which is a subclass of an abstract state machine class 312 period. The liver segmentation classes 300 also includes a merged polydata object class 315 of which a medical object resection planner feature class 318 is composed of, which is a subclass of the resection planner feature class 309.

The liver segmentation classes 300 also includes a visibility state class 321, which is a child class of a resection state class 324, which is a child class of an abstract state class 327, which is a child class of the abstract state machine class 312. A control point class 330 of which a cutting surface class 333 is composed of, of which a surfaces manager class 336 is composed of which, is a child class of a planning state class 339, which is a child class of the resection state class 324.

The liver segmentation classes 300 includes a liver resection tool class 342, which is a child class of the cutting surface class 333, and a vessel clip class 345 of which a clip manager class 348 is composed of. The clip manager class 348 is a child class of the planning state class 339. A cut request class 351 is a child class of a calculation state class 354, which is a child class of the resection state class 324. An object status service connection class 357 is a child class of the calculation state class 354. An except request class 360 is a child class of a review state class 363, which is a child class of the resection state class 324. A reject request class 366 is a child class of the review state class 363. A resection data container class 369 is a child class of the resection state class 324, and also a child class of the resection state machine class 372, which is a child class of the abstract state machine class 312. An instantiated object of the resection data container class 369 generates a liver resection object (such as 280 in FIG. 2). An abstract resection feature class 375 is a child class of the abstract state machine class 312.

### Method Implementations

In the previous section, apparatus of the operation of an implementation was described. In this section, the particular methods performed by apparatus of such an implementation are described by reference to a series of flowcharts.

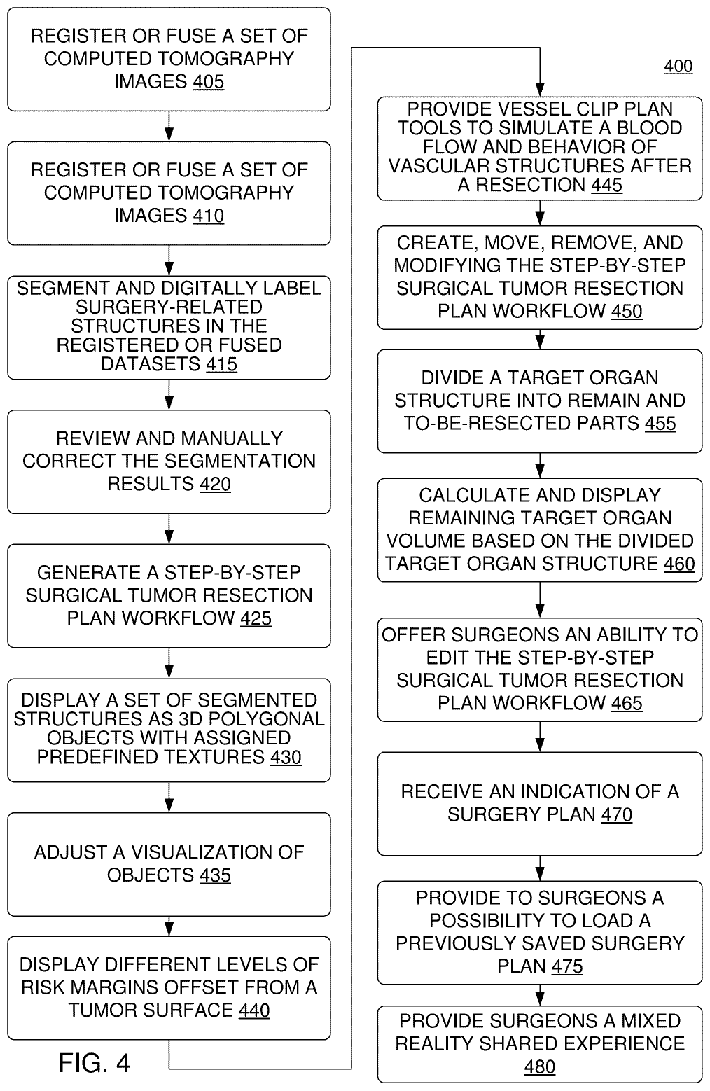

FIG. 4 is a flowchart of a method 400 of tumor resection planning in mixed reality, according to an implementation. In some implementations, method 400 is performed by apparatus 100 in FIG. 1.

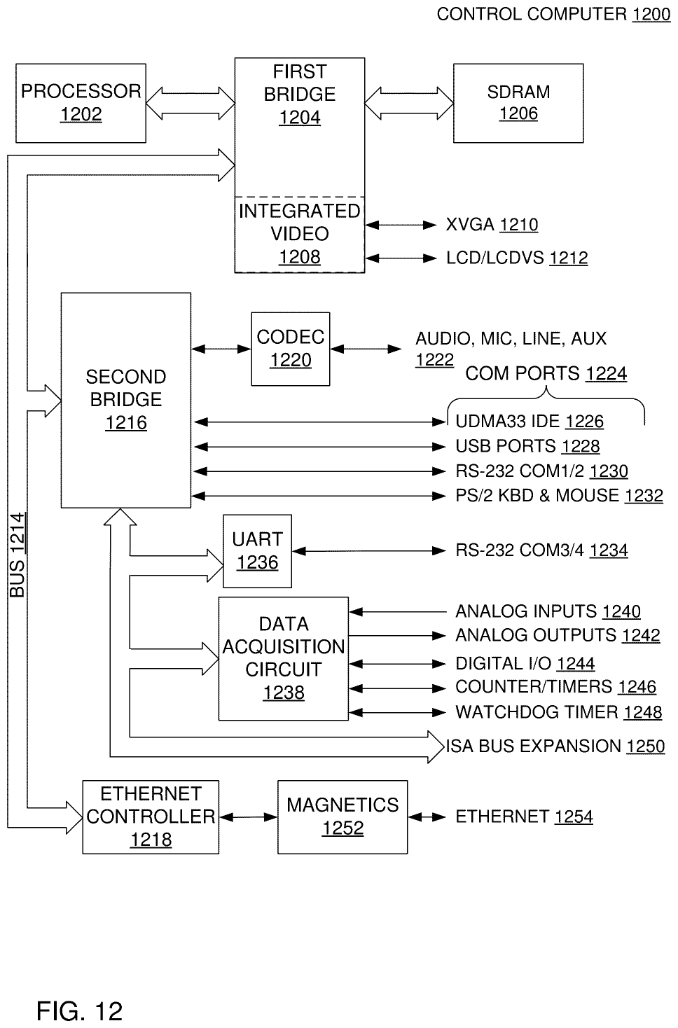

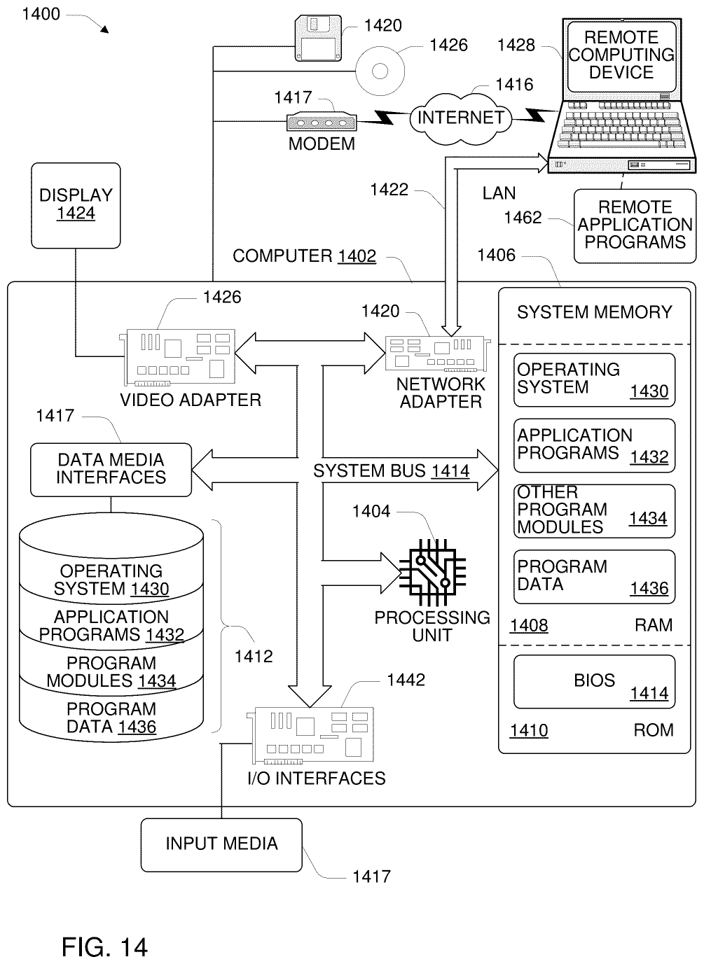

In some implementations, method 400 is implemented as a sequence of instructions which, when executed by a processor, such as processor 1202 in FIG. 12, processing unit 1404 in FIG. 14 or main processor 1502 cause the processor to perform the respective method. In other implementations, method 400 is implemented as a computer-accessible medium having executable instructions capable of directing a processor, such as processor 1202 in FIG. 12, processing unit 1404 in FIG. 14 or main processor 1502 to perform the respective method. In varying implementations, the medium is a magnetic medium, an electronic medium, or an optical medium.

Method 400 includes at block 405 registering or fusing a set of computed tomography images in different phases and/or registering computed tomography and magnetic resonance images, yielding registered or fused datasets, according to an implementation.

Method 400 also includes at block 410 navigating synchronously through slices in the registered or fused datasets, according to an implementation.

Method 400 also includes at block 415 segmenting and digitally labeled surgery-related structures in the registered or fused datasets. For example, in hepatectomy (Liver Resection) block 415 includes parenchyma, hepatic vein, portal vein, and tumors, from fused DICOMs, yielding segmentation results, according to an implementation.

Method 400 also includes at block 420 reviewing and manually correcting the segmentation results through a browser-based segmentation tool for a final clinical validation of the segmentation results, yielding reviewed and manually corrected segmentation results, according to an implementation.

Method 400 also includes at block 425 generating a step-by-step surgical tumor resection plan workflow that is adaptable to different organs such as a liver, pancreas, a lung, and a brain from the reviewed and manually corrected segmentation results, according to an implementation.

Method 400 also includes at block 430 displaying a set of segmented structures as 3D polygonal objects with assigned predefined textures to enhance a perception by a surgeon of relationships between anatomical structures in reference to the step-by-step surgical tumor resection plan workflow, yielding a displayed set of segmented structures, according to an implementation.

Method 400 also includes at block 435 adjusting a visualization of objects in terms of transparency and color to meet user's custom needs, yielding visually adjusted objects, according to an implementation.

Method 400 also includes at block 440 displaying different levels of risk margins offset from a tumor surface, with user-defined margin values that can be changed on demand, yielding displayed levels of risk margins offset from the tumor surface, according to an implementation.

Method 400 also includes at block 445 providing vessel clip plan tools to simulate a blood flow and behavior of vascular structures after a resection according to the step-by-step surgical tumor resection plan workflow, yielding provided vessel clip plan tools, according to an implementation.

Method 400 also includes at block 450 creating, moving, removing, and modifying the step-by-step surgical tumor resection plan workflow around a set of target tumor(s), yielding updated step-by-step surgical tumor resection plan, according to an implementation.

Method 400 also includes at block 455 dividing a target organ structure into remain and to-be-resected parts based on a user-defined resection plan(s) by apply cloud-based boolean mesh operations yielding a divided target organ structure, according to an implementation.

Method 400 also includes at block 460 calculating and displaying remaining target organ volume based on the divided target organ structure, yielding a calculated and displayed remaining target organ volume, according to an implementation.

Method 400 also includes at block 465 offering surgeons an ability to edit the step-by-step surgical tumor resection plan workflow and reapply the divided target organ structure in reference to the calculated and displayed remaining target organ volume, according to an implementation.

Method 400 also includes at block 470 receiving an indication of a surgery plan including divided polygonal objects, remaining organ volume, and vessel clip information, according to an implementation.

Method 400 also includes at block 475 providing surgeons an option to load a previously saved surgery plan, according to an implementation.

Method 400 also includes at block 480 providing surgeons a mixed reality shared experience to remotely discuss the surgery plan with other tumor board members.

Some implementations of method 400 further include applying a set of cloud-based boolean mesh operations to accurately divide the target organ structure into remain and to-be-resected parts based on the user-defined resection plan(s). Some implementations of method 400 a mixed reality technology comprises virtual reality (VR), augmented reality (AR), and holographic displays. Some implementations of method 400 further include registering computed tomography images in different phases and the magnetic resonance images to enable comprehensive understanding of tumor location and a relationship with surrounding structures. Some implementations of method 400 further include adjusting a transparency and a color of displaying objects to enhance a perception of the surgeon of anatomical structures. Some implementations of method 400 further include interactively adjusting risk margins offset from the tumor surface based on user preferences. Some implementations of method 400 further include simulating a blood flow and a behavior of vascular structures post-resection, aid in preoperative evaluation and risk assessment. Some implementations of method 400 further include creating, moving, removing, and modifying tumor resection plans to precisely define a resection area. Some implementations of method 400 further include saving and loading surgery plans to ensure continuity of care and facilitate modifications to the surgical plan. In some implementations of method 400, the mixed reality shared experience enables remote collaboration and discussion of the surgery plan among surgeons and members of a professional tumor board.

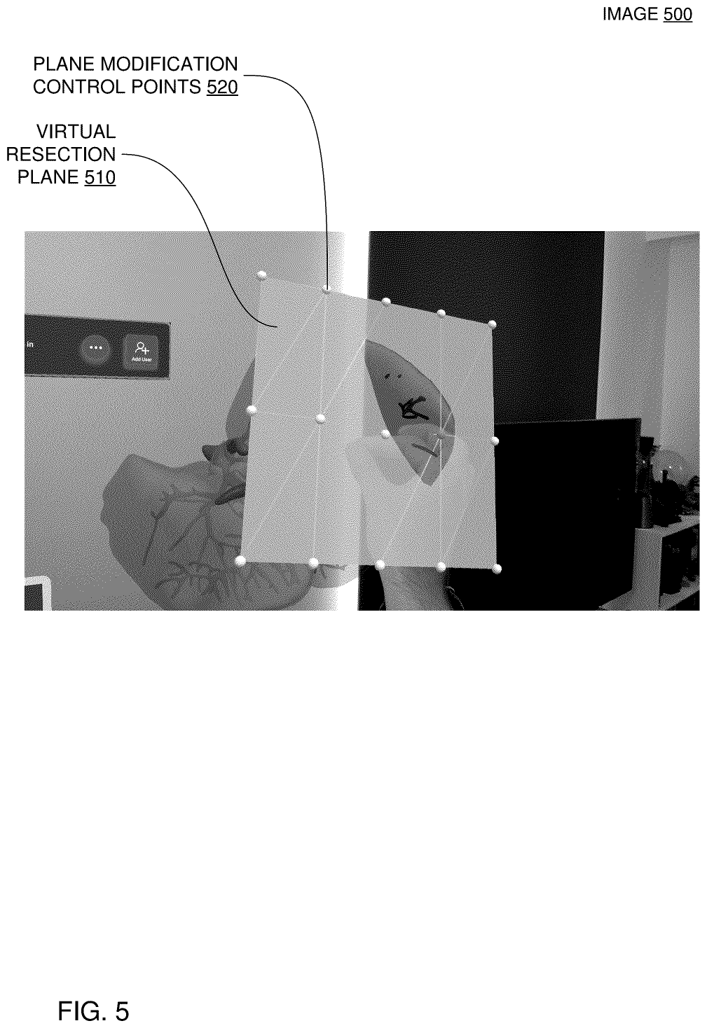

FIG. 5 is a sample photo representing the users field of view from a mixed reality scene consisting of a medical object hologram with an image 500 of a liver with a non-modified resection plane, according to an implementation. Image 500 has a virtual resection plane 510 that has 15 plane modification points 520.

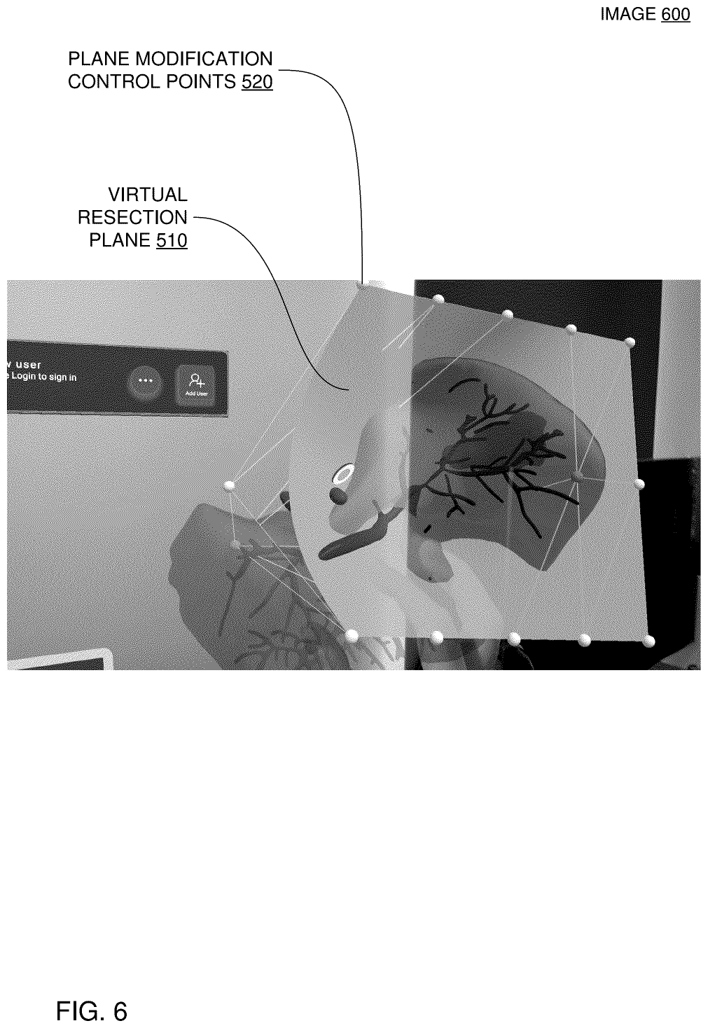

FIG. 6 is a sample photo representing the users field of view from a mixed reality scene consisting of a medical object hologram with an image 600 of a liver with a liver with a user defined resection plane, according to an implementation. Image 600 has the virtual resection plane 510 that has the 15 plane modification points 520.

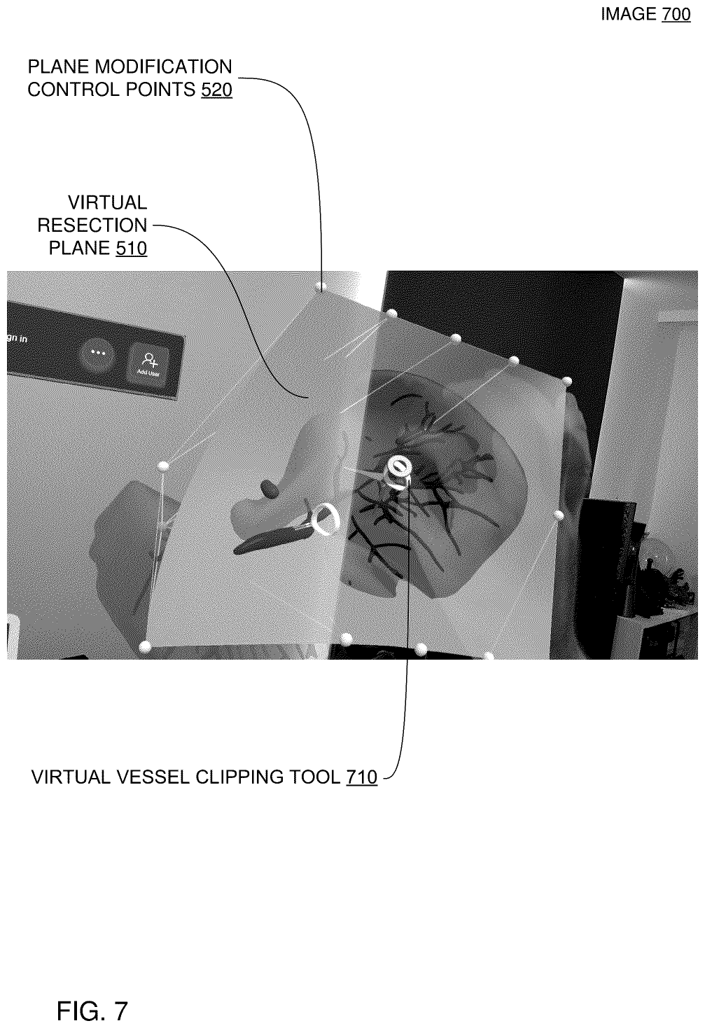

FIG. 7 is a sample photo representing the users field of view from a mixed reality scene consisting of a medical object hologram with an image 700 of a liver with virtual vessel clips, according to an implementation. Image 600 has the virtual resection plane 510 that has the 15 plane modification points 520 and a virtual vessel clipping tool 710.

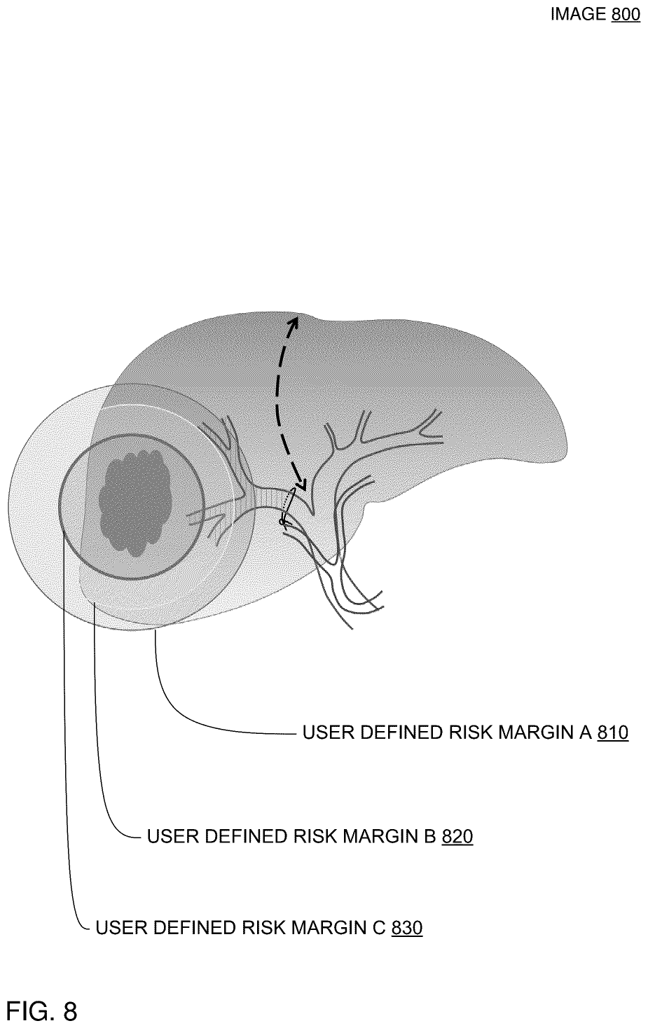

FIG. 8 is a diagram of an image 800 of a liver with user defined risk margins, according to an implementation. Image 800 includes user defined risk margin A 810, user defined risk margin B 820 and user defined risk margin C 830.

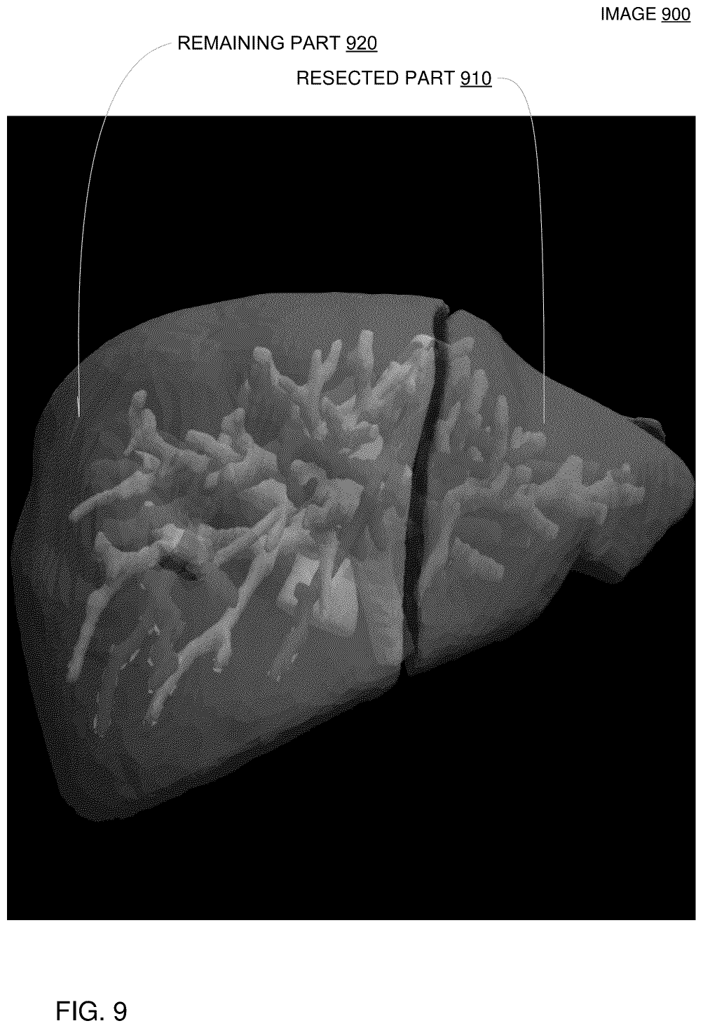

FIG. 9 is a diagram of an image 900 of a liver with resected and non-resected parts, to an implementation. Image 900 includes a resected part 910 and a remaining part 920 according that is non-resected.

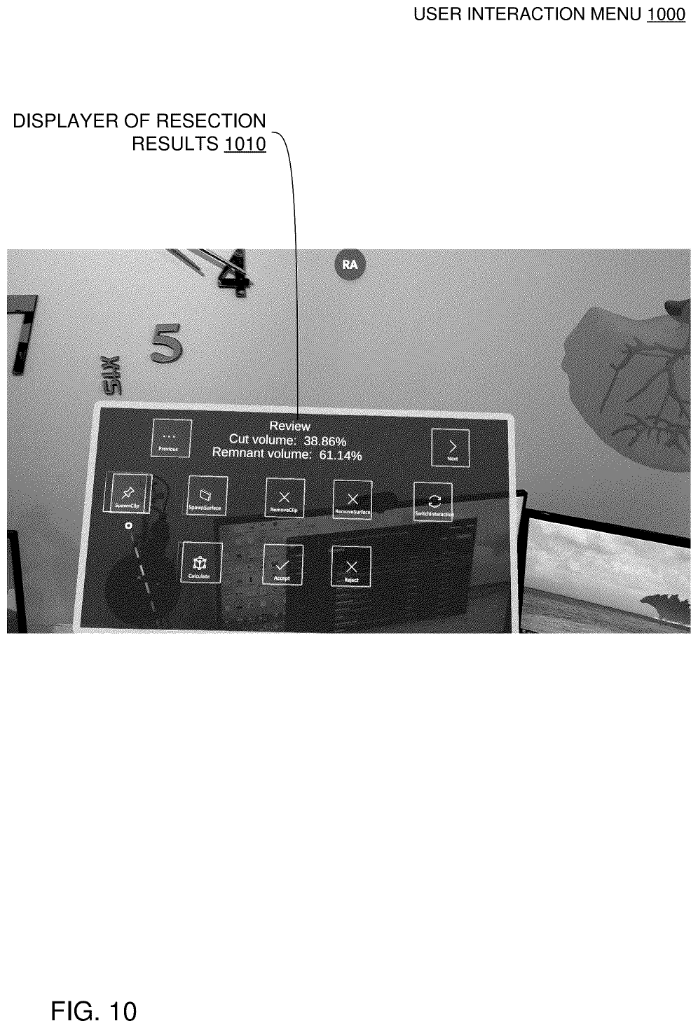

FIG. 10 is a sample photo representing the users field of view from a mixed reality scene consisting of a medical object hologram with a virtual user interaction menu 1000 with a result of resection, according to an implementation. User interaction menu 1000 includes a displayer of resection results 1010.

### Hardware and Operating Environment

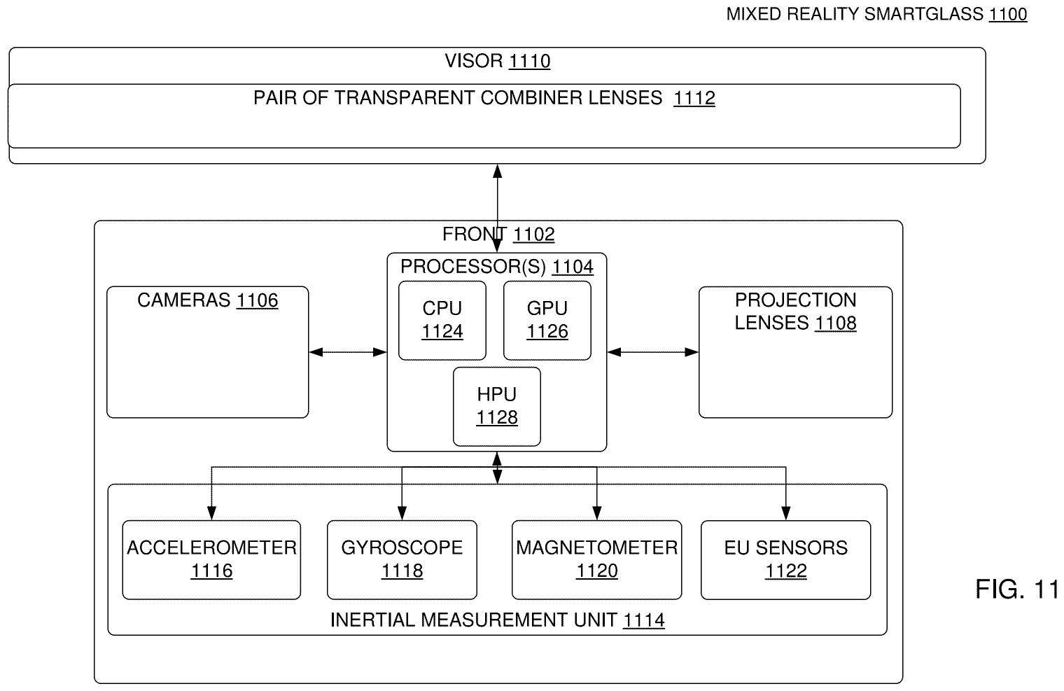

FIG. 11 is a block diagram of a mixed reality smartglass 1100 that is operable to conduct a virtual medical masterclass, according to an implementation. The mixed reality smartglass 1100 can include the apparatus 100 in FIG. 1 and apparatus 200 in FIG. 2 and/or can perform the method 400 in FIG. 4. The mixed reality smartglass 1100 is a head-mounted display unit and in some implementation is connected to an adjustable, cushioned inner headband, which can tilt the mixed reality smartglass 1100 up and down, as well as forward and backward. To wear the unit, the user can fit the mixed reality smartglass 1100 on their head, using an adjustment wheel at the back of the headband to secure the mixed reality smartglass 1100 around the crown, supporting and distributing the weight of the mixed reality smartglass 1100 equally for comfort, before tilting the visor towards the front of the eyes.

In some implementations, the front 1102 of the unit houses many of the sensors and related hardware, including the processors 1104, cameras 1106 and projection lenses 1108. In some implementations, the visor 1110 is tinted; enclosed in the visor 1110 is a pair of transparent combiner lenses 1112, in which the projected images are displayed in the lower half. In some implementations, the mixed reality smartglass 1100 must be calibrated to the interpupillary distance (IPD), or accustomed vision of the user.

In some implementations, along the bottom edges of the side, located near the user's ears, are a pair of small, 14D audio speakers. The speakers, competing against typical sound systems, do not obstruct external sounds, allowing the user to hear virtual sounds, along with the environment. Using head-related transfer functions, the mixed reality smartglass 1100 generates binaural audio, which can simulate spatial effects; meaning the user, virtually, can perceive and locate a sound, as though it is coming from a virtual pinpoint or location.

In some implementations, on the top edge are two pairs of buttons: display brightness buttons above the left ear, and volume buttons above the right ear. Adjacent buttons are shaped differently-one concave, one convex-so that the user can distinguish them by touch.

The mixed reality smartglass 1100 includes an inertial measurement unit (IMU) 1114 (which includes an accelerometer 1116, a gyroscope 1118, and a magnetometer 1120) four “environment understanding” (EU) sensors 1122 (two on each side), and in some implementations, an energy-efficient depth camera with a 120°×120° angle of view, a 2.4-megapixel photographic video camera, a four-microphone array, and an ambient light sensor.

In some implementations, SoC contains a CPU 1124 and a GPU 1126. In some implementations, the mixed reality smartglass 1100 features a custom-made holographic processing unit (HPU) 1128, a coprocessor manufactured specifically for the mixed reality smartglass 1100. In some implementations, the SoC and the HPU 1128 each have 1 GB LPDDR3 and share 8 MB SRAM, with the SoC also controlling 64 GB eMMC and running an operating system. In some implementations, the HPU 1128 uses 28 custom DSPs from Tensilica to process and integrates data from the sensors, as well as handling tasks such as spatial mapping, gesture recognition, and voice and speech recognition. In some implementations, the HPU 1128 processes “terabytes of information.” The display field of view can be 30°×17.5°. The HPU 2000 in FIG. 20 is one example of the HPU 1128.

The mixed reality smartglass 1100 can include IEEE 802.11ac Wi-Fi and Bluetooth 4.1 Low Energy (LE) wireless connectivity. The headset can use Bluetooth LE to pair with the included Clicker, a thumb-sized finger-operating input device that can be used for interface scrolling and selecting. The Clicker features a clickable surface for selecting, and an orientation sensor which provides for scrolling functions via tilting and panning of the unit. The Clicker features an elastic finger loop for holding the device, and a USB 2.0 micro-B receptacle for charging its internal battery.

FIG. 12 is a block diagram of a mixed reality tumor resection planning control computer 1200 in which different implementations can be practiced. The mixed reality tumor resection planning control computer 1200 includes a processor 1202 (such as a Pentium III processor from Intel Corp. in this example) which includes dynamic and static ram and non-volatile program read-only-memory (not shown), a first bridge 1204, operating memory 1206 (SDRAM in this example). The first bridge 1204 includes integrated video 1208 that couples the mixed reality tumor resection planning control computer 1200 to a XVGA communication path 1210 and a LCD and/or LCDVS device 1212.

The first bridge 1204 is operably coupled to a bus 1214 and the bus 1214 is operably coupled to a second bridge 1216 and an Ethernet® controller 1218.

The second bridge 1216 is operably coupled to a CODEC 1220 and the CODEC 1220 is coupled to an audio port 1222. The second bridge 1216 is operably coupled to communication ports 1224 (e.g., UDMA IDE 1226, USB port(s) 1228, RS-232 1230 COM1/2 and/or keyboard interface 1232).

An RS-232 port 1234 is coupled through a universal asynchronous receiver/transmitter (UART) 1236 to the second bridge 1216.

The second bridge 1216 is operably coupled to a data acquisition circuit 1238 with analog inputs 1240 and outputs 1242 and digital inputs and outputs 1244.

In some implementations of the mixed reality tumor resection planning control computer 1200, the data acquisition circuit 1238 is also coupled to counter timer ports 1246 and watchdog timer ports 1248. In some implementations of the mixed reality tumor resection planning control computer 1200, the second bridge 1216 is operably coupled to an expansion bus 1250.

In some implementations, the Ethernet® controller 1218 is operably coupled to magnetics 1252 which is operably coupled to an Ethernet® local area network 1254.

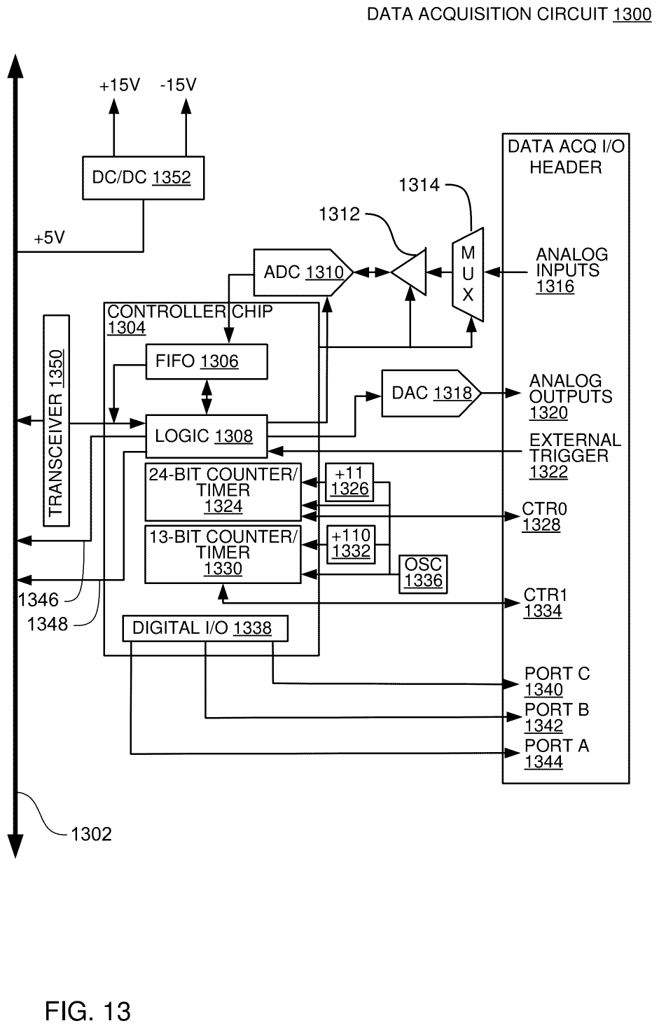

FIG. 13 is a block diagram of a data acquisition circuit 1300 of a mixed reality tumor resection planning control computer, according to an implementation. The data acquisition circuit 1300 is one example of the data acquisition circuit 1238 in FIG. 12 above. Some implementations of the data acquisition circuit 1300 provide 12-bit A/D performance with input voltage capability up to +/−10V, and programmable input ranges.

The data acquisition circuit 1300 can include a bus 1302, such as a conventional PC/104 bus. The data acquisition circuit 1300 can be operably coupled to a controller chip 1304. Some implementations of the controller chip 1304 include an analog/digital first-in/first-out (FIFO) buffer 1306 that is operably coupled to controller logic 1308. In some implementations of the data acquisition circuit 1300, the FIFO 1306 receives signal data from and analog/digital converter (ADC) 1310, which exchanges signal data with a programmable gain amplifier 1312, which receives data from a multiplexer 1314, which receives signal data from analog inputs 1316.

In some implementations of the data acquisition circuit 1300, the controller logic 1308 sends signal data to the ADC 1310 and a digital/analog converter (DAC) 1318. The DAC 1318 sends signal data to analog outputs. In some implementations of the data acquisition circuit 1300, the controller logic 1308 receives signal data from an external trigger 1322.

In some implementations of the data acquisition circuit 1300, the controller chip 1304 includes a digital input/output (I/O) component 1338 that sends digital signal data to computer output ports.

In some implementations of the data acquisition circuit 1300, the controller logic 1308 sends signal data to the bus 1302 via a control line 1346 and an interrupt line 1348. In some implementations of the data acquisition circuit 1300, the controller logic 1308 exchanges signal data to the bus 1302 via a transceiver 1350.

Some implementations of the data acquisition circuit 1300 include 16-bit D/A channels, programmable digital I/O lines, and programmable counter/timers. Analog circuitry can be placed away from the high-speed digital logic to ensure low-noise performance for important applications. Some implementations of the data acquisition circuit 1300 are fully supported by operating systems that can include, but are not limited to, DOS™, Linux™, RTLinux™, QNX™, Windows 108/NT/2000/XP/CE™, Forth™, and VxWorks™ to simplify application development.

FIG. 14 is a block diagram of a hardware and operating environment 1400 in which different implementations can be practiced. The description of FIG. 14 provides an overview of computer hardware and a suitable computing environment in conjunction with which some implementations can be implemented. Implementations are described in terms of a computer executing computer-executable instructions. However, some implementations can be implemented entirely in computer hardware in which the computer-executable instructions are implemented in read-only memory. Some implementations can also be implemented in client/server computing environments where remote devices that perform tasks are linked through a communications network. Program modules can be located in both local and remote memory storage devices in a distributed computing environment.

Computer 1402 includes a processing unit 1404, commercially available from Intel, Motorola, Cyrix and others. The computer 1402 also includes system memory 1406 that includes random-access memory RAM 1408 and read-only memory ROM 1410. The computer 1402 also includes one or more mass storage devices 1412; and a system bus 1414 that operatively couples various system components to the processing unit 1404. The RAM 1408 and ROM 1410, and mass storage devices 1412, are types of computer-accessible media. Mass storage devices 1412 are more specifically types of nonvolatile computer-accessible media and can include one or more hard disk drives, floppy disk drives, optical disk drives, and tape cartridge drives. The processing unit 1404 executes computer programs stored on the computer-accessible media.

Computer 1402 can be communicatively connected to the Internet 1416 via a communication device, such as modem 1418. Internet 1416 connectivity is well known within the art. In one implementation, the modem 1418 responds to communication drivers to connect to the Internet 1416 via what is known in the art as a “dial-up connection.” In another implementation, the communication device is an Ethernet® or network adapter 1420 connected to a local-area network (LAN) 1422 that itself is connected to the Internet 1416 via what is known in the art as a “direct connection” (e.g., T1 line, etc.).

A user enters commands and information into the computer 1402 through input devices such as a keyboard (not shown) or a pointing device (not shown). The keyboard permits entry of textual information into computer 1402, as known within the art, and implementations are not limited to any particular type of keyboard. Pointing device permits the control of the screen pointer provided by a graphical user interface (GUI) of operating systems such as versions of Microsoft Windows®. Implementations are not limited to any particular pointing device. Such pointing devices include mice, touch pads, trackballs, remote controls and point sticks. Other input devices (not shown) can include a microphone, joystick, game pad, satellite dish, scanner, or the like.

In some implementations, computer 1402 is operatively coupled to a display device 1424. Display device 1424 is connected to the system bus 1414 through a video adapter 1426. Display device 1424 permits the display of information, including computer, video and other information, for viewing by a user of the computer. Implementations are not limited to any particular display device 1424. Such display devices include cathode ray tube (CRT) displays (monitors), as well as flat panel displays such as liquid crystal displays (LCD's). In addition to a monitor, computers typically include other peripheral input/output devices such as printers (not shown). Speakers (not shown) provide audio output of signals. Speakers are also connected to the system bus 1414.

Computer 1402 can be operated using at least one operating system to provide a graphical user interface (GUI) including a user-controllable pointer. Computer 1402 can have at least one web browser application program executing within at least one operating system, to permit users of computer 1402 to access intranet or Internet world-wide-web pages as addressed by Universal Resource Locator (URL) addresses. Examples of browser application programs include Netscape Navigator® and Microsoft Internet Explorer®.

The computer 1402 can operate in a networked environment using logical connections to one or more remote computers, such as remote computer 1428. These logical connections are achieved by a communication device coupled to, or a part of, the computer 1402. Implementations are not limited to a particular type of communications device. The remote computer 1428 can be another computer, a server, a router, a network PC, a client, a peer device or other common network node. The logical connections depicted in FIG. 14 include the local-area network (LAN) 1422 and a wide-area network (WAN). Such networking environments are commonplace in offices, enterprise-wide computer networks, intranets and the Internet.

When used in a LAN-networking environment, the computer 1402 and remote computer 1428 are connected to the local network 1422 through network interfaces or adapters 1420, which is one type of communications device 1418. When used in a conventional WAN-networking environment, the computer 1402 and remote computer 1428 communicate with a WAN through modems. The modems, which can be internal or external, is connected to the system bus 1414. In a networked environment, program modules depicted relative to the computer 1402, or portions thereof, can be stored in the remote computer 1428.

Computer 1402 also includes an operating system 1430 that can be stored on the RAM 1408 and ROM 1410, and/or mass storage device 1412, and is and executed by the processing unit 1404. Examples of operating systems include Microsoft Windows®, Apple MacOS®, Linux®, UNIX®, providing capability for supporting application programs 1432 using, for example, code modules written in the C++® computer programming language. Examples are not limited to any particular operating system, however, and the construction and use of such operating systems are well known within the art.

Instructions can be stored via the mass storage devices 1412 or system memory 1406, including one or more application programs 1432, other program modules 1434 and program data 1436.

Computer 1402 also includes power supply. Each power supply can be a battery.

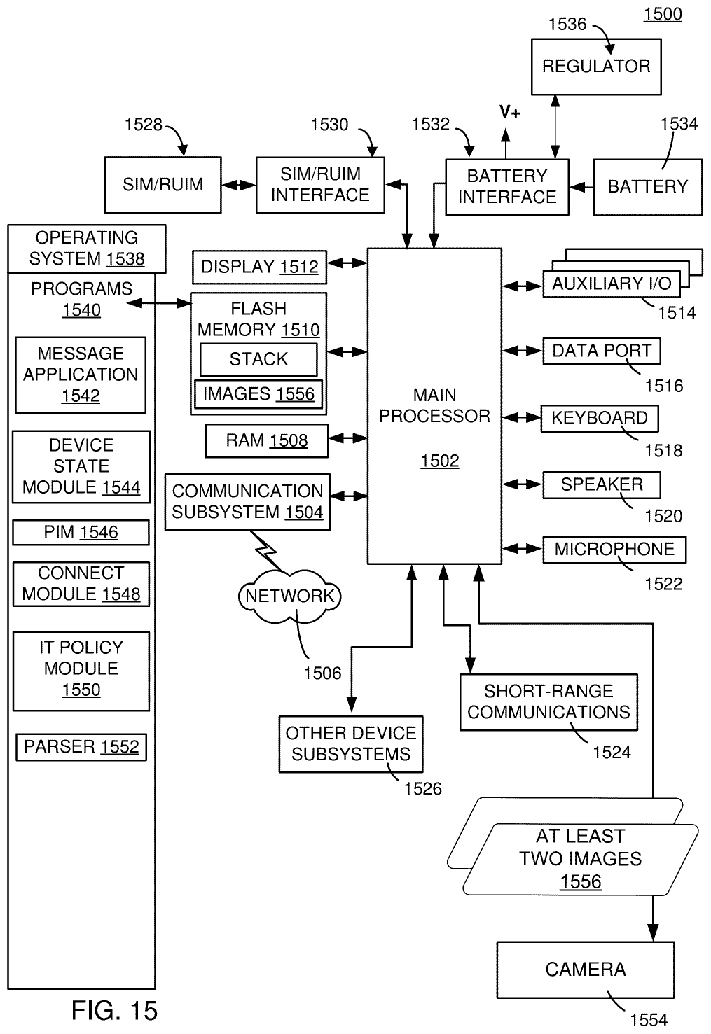

FIG. 15 is a block diagram of a mixed reality tumor resection planning control mobile device 1500, according to an implementation. The mixed reality tumor resection planning control mobile device 1500 includes a number of components such as a main processor 1502 that controls the overall operation of the mixed reality tumor resection planning control mobile device 1500. Communication functions, including data and voice communications, are performed through a communication subsystem 1504. The communication subsystem 1504 receives messages from and sends messages to a wireless network 1506. In this exemplary implementation of the mixed reality tumor resection planning control mobile device 1500, the communication subsystem 1504 is configured in accordance with the Global System for Mobile Communication (GSM), General Packet Radio Services (GPRS) standards, 3G, 4G, 5G and/or 6G. It will also be understood by persons skilled in the art that the implementations described herein are intended to use any other suitable standards that are developed in the future. The wireless link connecting the communication subsystem 1504 with the wireless network 1506 represents one or more different Radio Frequency (RF) channels, operating according to defined protocols specified for 4G or 5G communications. With newer network protocols, these channels are capable of supporting both circuit switched voice communications and packet switched data communications.

Although the wireless network 1506 associated with mixed reality tumor resection planning control mobile device 1500 is a GSM/GPRS, 3G, 4G, 5G and/or 6G wireless network in one exemplary implementation, other wireless networks may also be associated with the mixed reality tumor resection planning control mobile device 1500 in variant implementations. The different types of wireless networks that may be employed include, for example, data-centric wireless networks, voice-centric wireless networks, and dual-mode networks that can support both voice and data communications over the same physical base stations. Combined dual-mode networks include, but are not limited to, Code Division Multiple Access (CDMA) or CDMA2000 networks, GSM/GPRS networks, 3G, 4G, 5G and/or 6G. Some other examples of data-centric networks include WiFi 802.11, Mobitex™ and DataTAC™ network communication systems. Examples of other voice-centric data networks include Personal Communication Systems (PCS) networks like GSM and Time Division Multiple Access (TDMA) systems.

The main processor 1502 also interacts with additional subsystems such as a Random Access Memory (RAM) 1508, a flash memory 1510, a display 1512, an auxiliary input/output (I/O) subsystem 1514, a data port 1516, a keyboard 1518, a speaker 1520, a microphone 1522, short-range communications 1524 and other device subsystems 1526.

Some of the subsystems of the mixed reality tumor resection planning control mobile device 1500 perform communication-related functions, whereas other subsystems may provide “resident” or on-device functions. By way of example, the display 1512 and the keyboard 1518 may be used for both communication-related functions, such as entering a text message for transmission over the wireless network 1506, and device-resident functions such as a calculator or task list.

The mixed reality tumor resection planning control mobile device 1500 can send and receive communication signals over the wireless network 1506 after required network registration or activation procedures have been completed. Network access is associated with a subscriber or user of the mixed reality tumor resection planning control mobile device 1500. To identify a subscriber, the mixed reality tumor resection planning control mobile device 1500 requires a SIM/RUIM card 1528 (i.e. Subscriber Identity Module or a Removable User Identity Module) to be inserted into a SIM/RUIM interface 1530 in order to communicate with a network. The SIM card or RUIM 1528 is one type of a conventional “smart card” that can be used to identify a subscriber of the mixed reality tumor resection planning control mobile device 1500 and to customize the mixed reality tumor resection planning control mobile device 1500, among other aspects. Without the SIM card 1528, the mixed reality tumor resection planning control mobile device 1500 is not fully operational for communication with the wireless network 1506. By inserting the SIM card/RUIM 1528 into the SIM/RUIM interface 1530, a subscriber can access all subscribed services. Services may include: web browsing and messaging such as e-mail, voice mail, Short Message Service (SMS), and Multimedia Messaging Services (MMS). More advanced services may include: point of sale, field service and sales force automation. The SIM card/RUIM 1528 includes a processor and memory for storing information. Once the SIM card/RUIM 1528 is inserted into the SIM/RUIM interface 1530, it is coupled to the main processor 1502. In order to identify the subscriber, the SIM card/RUIM 1528 can include some user parameters such as an International Mobile Subscriber Identity (IMSI). An advantage of using the SIM card/RUIM 1528 is that a subscriber is not necessarily bound by any single physical mobile device. The SIM card/RUIM 1528 may store additional subscriber information for a mobile device as well, including datebook (or calendar) information and recent call information. Alternatively, user identification information can also be programmed into the flash memory 1510.

The mixed reality tumor resection planning control mobile device 1500 is a battery-powered device and includes a battery interface 1532 for receiving one or more rechargeable batteries 1534. In one or more implementations, the battery 1534 can be a smart battery with an embedded microprocessor. The battery interface 1532 is coupled to a regulator 1536, which assists the battery 1534 in providing power V+ to the mixed reality tumor resection planning control mobile device 1500. Although current technology makes use of a battery, future technologies such as micro fuel cells may provide the power to the mixed reality tumor resection planning control mobile device 1500.

The mixed reality tumor resection planning control mobile device 1500 also includes an operating system 1538 and software components 1540 to 1552 which are described in more detail below. The operating system 1538 and the software components 1540 to 1552 that are executed by the main processor 1502 are typically stored in a persistent store such as the flash memory 1510, which may alternatively be a read-only memory (ROM) or similar storage element (not shown). Those skilled in the art will appreciate that portions of the operating system 1538 and the software components 1540 to 1552, such as specific device applications, or parts thereof, may be temporarily loaded into a volatile store such as the RAM 1508. Other software components can also be included.

The subset of software components 1540 that control basic device operations, including data and voice communication applications, will normally be installed on the mixed reality tumor resection planning control mobile device 1500 during its manufacture. Other software applications include a message application 1542 that can be any suitable software program that allows a user of the mixed reality tumor resection planning control mobile device 1500 to send and receive electronic messages. Various alternatives exist for the message application 1542 as is well known to those skilled in the art. Messages that have been sent or received by the user are typically stored in the flash memory 1510 of the mixed reality tumor resection planning control mobile device 1500 or some other suitable storage element in the mixed reality tumor resection planning control mobile device 1500. In one or more implementations, some of the sent and received messages may be stored remotely from the mixed reality tumor resection planning control mobile device 1500 such as in a data store of an associated host system with which the mixed reality tumor resection planning control mobile device 1500 communicates.

The software applications can further include a device state module 1544, a Personal Information Manager (PIM) 1546, and other suitable modules (not shown). The device state module 1544 provides persistence, i.e. the device state module 1545 ensures that important device data is stored in persistent memory, such as the flash memory 1510, so that the data is not lost when the mixed reality tumor resection planning control mobile device 1500 is turned off or loses power.

The PIM 1546 includes functionality for organizing and managing data items of interest to the user, such as, but not limited to, e-mail, contacts, calendar events, voice mails, appointments, and task items. A PIM application has the ability to send and receive data items via the wireless network 1506. PIM data items may be seamlessly integrated, synchronized, and updated via the wireless network 1506 with the mobile device subscriber's corresponding data items stored and/or associated with a host computer system. This functionality creates a mirrored host computer on the mixed reality tumor resection planning control mobile device 1500 with respect to such items. This can be particularly advantageous when the host computer system is the mobile device subscriber's office computer system.

The connect module 1548 includes a set of APIs that can be integrated with the mixed reality tumor resection planning control mobile device 1500 to allow the mixed reality tumor resection planning control mobile device 1500 to use any number of services associated with the enterprise system. The connect module 1548 allows the mixed reality tumor resection planning control mobile device 1500 to establish an end-to-end secure, authenticated communication pipe with the host system. A subset of applications for which access is provided by the connect module 1548 can be used to pass IT policy commands from the host system to the mixed reality tumor resection planning control mobile device 1500. This can be done in a wireless or wired manner. These instructions can then be passed to the IT policy module 1550 to modify the configuration of the mixed reality tumor resection planning control mobile device 1500. Alternatively, in some cases, the IT policy update can also be done over a wired connection.

The IT policy module 1550 receives IT policy data that encodes the IT policy. The IT policy module 1550 then ensures that the IT policy data is authenticated by the mixed reality tumor resection planning control mobile device 1500. The IT policy data can then be stored in the flash memory 1510 in its native form. After the IT policy data is stored, a global notification can be sent by the IT policy module 1550 to all of the applications residing on the mixed reality tumor resection planning control mobile device 1500. Applications for which the IT policy may be applicable then respond by reading the IT policy data to look for IT policy rules that are applicable.

The IT policy module 1550 can include a parser 1552, which can be used by the applications to read the IT policy rules. In some cases, another module or application can provide the parser. Grouped IT policy rules, described in more detail below, are retrieved as byte streams, which are then sent (recursively) into the parser to determine the values of each IT policy rule defined within the grouped IT policy rule. In one or more implementations, the IT policy module 1550 can determine which applications are affected by the IT policy data and send a notification to only those applications. In either of these cases, for applications that are not being executed by the main processor 1502 at the time of the notification, the applications can call the parser or the IT policy module 1550 when they are executed to determine if there are any relevant IT policy rules in the newly received IT policy data.

All applications that support rules in the IT Policy are coded to know the type of data to expect. For example, the value that is set for the “WEP User Name” IT policy rule is known to be a string; therefore the value in the IT policy data that corresponds to this rule is interpreted as a string. As another example, the setting for the “Set Maximum Password Attempts” IT policy rule is known to be an integer, and therefore the value in the IT policy data that corresponds to this rule is interpreted as such.

After the IT policy rules have been applied to the applicable applications or configuration files, the IT policy module 1550 sends an acknowledgement back to the host system to indicate that the IT policy data was received and successfully applied.

Other types of software applications can also be installed on the mixed reality tumor resection planning control mobile device 1500. These software applications can be third party applications, which are added after the manufacture of the mixed reality tumor resection planning control mobile device 1500. Examples of third party applications include games, calculators, utilities, etc.

The additional applications can be loaded onto the mixed reality tumor resection planning control mobile device 1500 through at least one of the wireless network 1506, the auxiliary I/O subsystem 1514, the data port 1516, the short-range communications subsystem 1524, or any other suitable device subsystem 1524. This flexibility in application installation increases the functionality of the mixed reality tumor resection planning control mobile device 1500 and may provide enhanced on-device functions, communication-related functions, or both. For example, secure communication applications may enable electronic commerce functions and other such financial transactions to be performed using the mixed reality tumor resection planning control mobile device 1500.

The data port 1516 enables a subscriber to set preferences through an external device or software application and extends the capabilities of the mixed reality tumor resection planning control mobile device 1500 by providing for information or software downloads to the mixed reality tumor resection planning control mobile device 1500 other than through a wireless communication network. The alternate download path may, for example, be used to load an encryption key onto the mixed reality tumor resection planning control mobile device 1500 through a direct and thus reliable and trusted connection to provide secure device communication.

The data port 1516 can be any suitable port that enables data communication between the mixed reality tumor resection planning control mobile device 1500 and another computing device. The data port 1516 can be a serial or a parallel port. In some instances, the data port 1516 can be a USB port that includes data lines for data transfer and a supply line that can provide a charging current to charge the battery 1534 of the mixed reality tumor resection planning control mobile device 1500.

The short-range communications subsystem 1524 provides for communication between the mixed reality tumor resection planning control mobile device 1500 and different systems or devices, without the use of the wireless network 1506. For example, the subsystem 1524 may include an infrared device and associated circuits and components for short-range communication. Examples of short-range communication standards include standards developed by the Infrared Data Association (IrDA), Bluetooth, and the 802.11 family of standards developed by IEEE.

In use, a received signal such as a text message, an e-mail message, or web page download will be processed by the communication subsystem 1504 and input to the main processor 1502. The main processor 1502 will then process the received signal for output to the display 1512 or alternatively to the auxiliary I/O subsystem 1514. A subscriber may also compose data items, such as e-mail messages, for example, using the keyboard 1518 in conjunction with the display 1512 and possibly the auxiliary I/O subsystem 1514. The auxiliary subsystem 1514 may include devices such as: a touch screen, mouse, track ball, infrared fingerprint detector, or a roller wheel with dynamic button pressing capability. The keyboard 1518 is preferably an alphanumeric keyboard and/or telephone-type keypad. However, other types of keyboards may also be used. A composed item may be transmitted over the wireless network 1506 through the communication subsystem 1504.

For voice communications, the overall operation of the mixed reality tumor resection planning control mobile device 1500 is substantially similar, except that the received signals are output to the speaker 1520, and signals for transmission are generated by the microphone 1522. Alternative voice or audio I/O subsystems, such as a voice message recording subsystem, can also be implemented on the mixed reality tumor resection planning control mobile device 1500. Although voice or audio signal output is accomplished primarily through the speaker 1520, the display 1512 can also be used to provide additional information such as the identity of a calling party, duration of a voice call, or other voice call related information.

In some implementations, the mixed reality tumor resection planning control mobile device 1500 includes a camera 1554 receiving a plurality of images 1556 from and examining pixel-values of the plurality of images 1556.

1. A tumor resection planning tool in mixed reality, comprising:
a component having a computer-accessible medium having executable instructions capable of directing a processor to register or fuse a set of computed tomography images in different phases and/or register computed tomography and magnetic resonance images;
a component having the computer-accessible medium having executable instructions capable of directing the processor to synchronously navigate through slices in fused datasets, the component having the computer-accessible medium having executable instructions capable of directing the processor to synchronously navigate being operably coupled to the component having the computer-accessible medium having executable instructions capable of directing the processor to register or fuse the set of computed tomography images;
a component having the computer-accessible medium having executable instructions capable of directing the processor to segment and digitally label surgery-related structures, from fused DICOMs, yielding segmentation results, the component having the computer-accessible medium having executable instructions capable of directing the processor to segment being operably coupled to the component having the computer-accessible medium having executable instructions capable of directing the processor to synchronously navigate;
a component having the computer-accessible medium having executable instructions capable of directing the processor to review and manually correct the segmentation results through a browser-based segmentation tool for a final clinical validation of the segmentation results, the component having the computer-accessible medium having executable instructions capable of directing the processor to review and manually correct segmentation results being operably coupled to the component having the computer-accessible medium having executable instructions capable of directing the processor to segment;
a component having the computer-accessible medium having executable instructions capable of directing the processor to provide a step-by-step surgical tumor resection plan workflow that is adaptable to different organs such as a liver, pancreas, a lung, and a brain, the component having the computer-accessible medium having executable instructions capable of directing the processor to provide the step-by-step surgical tumor resection plan workflow being operably coupled to the component having the computer-accessible medium having executable instructions capable of directing the processor to review and manually correct segmentation results;
a component having the computer-accessible medium having executable instructions capable of directing the processor to display a set of segmented structures as 3D polygonal objects with assigned predefined textures to enhance a perception by a surgeon of relationships between anatomical structures, the component having the computer-accessible medium having executable instructions capable of directing the processor to display the set of the segmented structures being operably coupled to the component having the computer-accessible medium having executable instructions capable of directing the processor to provide the step-by-step surgical tumor resection plan workflow;
a component having the computer-accessible medium having executable instructions capable of directing the processor to adjust a visualization of objects in terms of transparency and color to meet user's custom needs, the component having the computer-accessible medium having executable instructions capable of directing the processor to adjust the visualization of the objects being operable to the component having the computer-accessible medium having executable instructions capable of directing the processor to display the set of segmented structures;
a component having the computer-accessible medium having executable instructions capable of directing the processor to display different levels of risk margins offset from a tumor surface, with user-defined margin values that can be changed on demand, the component having the computer-accessible medium having executable instructions capable of directing the processor to display the different levels of the risk margins being operably coupled to the component having the computer-accessible medium having executable instructions capable of directing the processor to adjust the visualization of objects;
a component having the computer-accessible medium having executable instructions capable of directing the processor to provide vessel clip plan tools to simulate a blood flow and behavior of vascular structures after a resection according to the step-by-step surgical tumor resection plan workflow, the component having the computer-accessible medium having executable instructions capable of directing the processor to provide vessel clip plan tools being operably coupled to the component having the computer-accessible medium having executable instructions capable of directing the processor to display the different levels of the risk margins;
a component having the computer-accessible medium having executable instructions capable of directing the processor to create, move, remove, and modify the step-by-step surgical tumor resection plan workflow around a set of target tumor(s), the component having the computer-accessible medium having executable instructions capable of directing the processor to creating, moving, removing, and modify step-by-step surgical tumor resection plan being operably coupled to the component having the computer-accessible medium having executable instructions capable of directing the processor to provide vessel clip plan tools;
a component having the computer-accessible medium having executable instructions capable of directing the processor to divide a target organ structure into remain and to-be-resected parts based on a user-defined resection plan(s) by applying cloud-based boolean mesh operations yielding a division, the component having the computer-accessible medium having executable instructions capable of directing the processor to divide a target organ structure being operably coupled to the component having the computer-accessible medium having executable instructions capable of directing the processor to create, move, remove, and modify the tumor resection plan;
a component having the computer-accessible medium having executable instructions capable of directing the processor to calculate and display remaining target organ volume based on the division, the component having the computer-accessible medium having executable instructions capable of directing the processor to calculate and display remaining target organ volume being operably coupled to the component having the computer-accessible medium having executable instructions capable of directing the processor to divide the target organ structure
a component having the computer-accessible medium having executable instructions capable of directing the processor to offer an ability to edit the step-by-step surgical tumor resection plan workflow and reapply the division, the component having the computer-accessible medium having executable instructions capable of directing the processor to offer an ability to edit the step-by-step surgical tumor resection plan workflow being operably coupled to the component having the computer-accessible medium having executable instructions capable of directing the processor to calculate and display remaining target organ volume;
a component having the computer-accessible medium having executable instructions capable of directing the processor to indicate a surgery plan includes divided polygonal objects, remain organ volume, and vessel clip information, the component having the computer-accessible medium having executable instructions capable of directing the processor to indicate a surgery plan being operably coupled to the component having the computer-accessible medium having executable instructions capable of directing the processor to offer the ability to edit the step-by-step surgical tumor resection plan workflow;
a component having the computer-accessible medium having executable instructions capable of directing the processor to provide an option to load a previously saved surgery plan, the component having the computer-accessible medium having executable instructions capable of directing the processor to provide an option to load a previously saved surgery plan being operably coupled to the component having the computer-accessible medium having executable instructions capable of directing the processor to indicate the surgery plan; and
a component having the computer-accessible medium having executable instructions capable of directing the processor to provide a mixed reality shared experience to remotely discuss the surgery plan with other tumor board members, the component having the computer-accessible medium having executable instructions capable of directing the processor to provide a mixed reality shared experience being operably coupled to the component having the computer-accessible medium having executable instructions capable of directing the processor to provide the option to load the previously saved surgery plan.

2. The tumor resection plan tool according to claim 1, further comprising apparatus having the computer-accessible medium having executable instructions capable of directing the processor to apply a set of cloud-based boolean mesh operations to accurately divide the target organ structure into remain and to-be-resected parts based on the user-defined resection plan(s).

3. The tumor resection plan tool according to claim 1, wherein a mixed reality technology comprises virtual reality (VR), augmented reality (AR), and holographic displays.

4. The tumor resection plan tool according to claim 1, further comprising a component having the computer-accessible medium having executable instructions capable of directing the processor to register computed tomography images in different phases and the magnetic resonance images to enable comprehensive understanding of tumor location and a relationship with surrounding structures.

5. The tumor resection planning tool according to claim 1, further comprising a component having the computer-accessible medium having executable instructions capable of directing the processor to adjust a transparency and a color of displayed objects to enhance a perception of the surgeon of anatomical structures.

6. The tumor resection planning tool according to claim 1, further comprising a component having the computer-accessible medium having executable instructions capable of directing the processor to interactively adjust risk margins offset from the tumor surface based on user preferences.

7. The tumor resection planning tool according to claim 1, further comprising a component having the computer-accessible medium having executable instructions capable of directing the processor to simulate a blood flow and a behavior of vascular structures after the resection, aid in preoperative evaluation and risk assessment.

8. The tumor resection planning tool according to claim 1, further comprising a component having the computer-accessible medium having executable instructions capable of directing the processor to create, move, remove, and modify tumor resection plans to precisely define a resection area.

9. The tumor resection planning tool according to claim 1, further comprising a component having the computer-accessible medium having executable instructions capable of directing the processor to save and load surgery plans to ensure continuity of care and facilitate modifications to the surgical plan.

10. The tumor resection planning tool according to claim 1, wherein the mixed reality shared experience enables remote collaboration and discussion of the surgery plan among and members of a professional tumor board.

11. A tumor resection planning tool in mixed reality, comprising:
a microprocessor that is configured to:
register or fuse a set of computed tomography images in different phases and/or register computed tomography and magnetic resonance images;
synchronously navigate through slices in fused datasets;
segment and digitally label surgery-related structures, from fused DICOMs, yielding segmentation results, and to synchronously navigate;
review and manually correct the segmentation results through a browser-based segmentation tool for a final clinical validation of the segmentation results,
provide a step-by-step surgical tumor resection plan workflow that is adaptable to different organs such as a liver, pancreas, a lung, and a brain;
display a set of segmented structures as 3D polygonal objects with assigned predefined textures to enhance a perception by a surgeon of relationships between anatomical structures;
adjust a visualization of objects in terms of transparency and color to meet user's custom needs;
display different levels of risk margins offset from a tumor surface, with user-defined margin values that can be changed on demand, the apparatus that is operable to display the different levels of the risk margins being operably coupled to the apparatus that is operable to adjust the visualization of objects;
provide vessel clip plan tools to simulate a blood flow and behavior of vascular structures after a resection according to the step-by-step surgical tumor resection plan workflow, the apparatus that is operable to provide vessel clip plan tools being operably coupled to the apparatus that is operable to display the different levels of the risk margins;
create, move, remove, and modify the step-by-step surgical tumor resection plan workflow around a set of target tumor(s);
divide a target organ structure into remain and to-be-resected parts based on a user-defined resection plan(s) by applying cloud-based boolean mesh operations yielding a division;
calculate and display remaining target organ volume based on the division;
offer an ability to edit the step-by-step surgical tumor resection plan workflow and reapply the division;
indicate a surgery plan includes divided polygonal objects, remain organ volume, and vessel clip information;
provide an option to load a previously saved surgery plan; and
provide a mixed reality shared experience to remotely discuss the surgery plan with other tumor board members.

12. The tumor resection plan tool according to claim 11, wherein the microprocessor is further configured to apply a set of cloud-based boolean mesh operations to accurately divide the target organ structure into remain and to-be-resected parts based on the user-defined resection plan(s).

13. The tumor resection plan tool according to claim 11, wherein a mixed reality technology comprises virtual reality (VR), augmented reality (AR), and holographic displays.

14. The tumor resection plan tool according to claim 11, wherein the microprocessor is further configured to register computed tomography images in different phases and the magnetic resonance images to enable comprehensive understanding of tumor location and a relationship with surrounding structures.

15. The tumor resection planning tool according to claim 11, wherein the microprocessor is further configured to adjust a transparency and a color of displayed objects to enhance a perception of the surgeon of anatomical structures.

16. The tumor resection planning tool according to claim 11, wherein the microprocessor is further configured to interactively adjust risk margins offset from the tumor surface based on user preferences.

17. The tumor resection planning tool according to claim 11, wherein the microprocessor is further configured to simulate a blood flow and a behavior of vascular structures after the resection, aid in preoperative evaluation and risk assessment.

18. The tumor resection planning tool according to claim 11, wherein the microprocessor is further configured to create, move, remove, and modify tumor resection plans to precisely define a resection area.

19. The tumor resection planning tool according to claim 11, wherein the microprocessor is further configured to save and load surgery plans to ensure continuity of care and facilitate modifications to the surgical plan.

20. The tumor resection planning tool according to claim 11, wherein the mixed reality shared experience enables remote collaboration and discussion of the surgery plan among and members of a professional tumor board.
